SEQUENCE LISTING (78 sequences)

SEQ ID NO: 1 - DNA, 94 bp, Human Immunodeficiency Virus
tagctcatgttgctaggcaactaaaccgcaatatcctgtagttcctcatttgcgttccgcatttgtgacgtgttagttcctgtttttacagtat

SEQ ID NO: 2 - DNA, 26 bp, Human Immunodeficiency Virus
tagctcatgttgctaggcaactaaac

SEQ ID NO: 3 - DNA, 18 bp, Human Immunodeficiency Virus
aggcaactaaaccgcaat

SEQ ID NO: 4 - DNA, 26 bp, Human Immunodeficiency Virus
ctaaaccgcaatatcctgtagttcct

SEQ ID NO: 5 - DNA, 31 bp, Human Immunodeficiency Virus
ccgcaatatcctgtagttcctcaatatagtt

SEQ ID NO: 6 - DNA, 23 bp, Human Immunodeficiency Virus
agttcctcatttgcgttccgcat

SEQ ID NO: 7 - DNA, 23 bp, Human Immunodeficiency Virus
tcatttgcgttccgcatttgtga

SEQ ID NO: 8 - DNA, 25 bp, Human Immunodeficiency Virus
gcgttccgcatttgtgacgtgttaa

SEQ ID NO: 9 - DNA, 21 bp, Human Immunodeficiency Virus
ttgtgacgtgttaagttcctg

SEQ ID NO: 10 - DNA, 23 bp, Human Immunodeficiency Virus
ttaagttcgtgtttttacagtat

SEQ ID NO: 11 - DNA, 80 bp, Human Immunodeficiency Virus
gttgctaggcaactaaaccgcaatatcctgtagttcctcatttgcgttccgcatttgtgacgtgttaagttcctgttttt

SEQ ID NO: 12 - DNA, 72 bp, Human Immunodeficiency Virus
tacaagggactttccgctggggactttccagggaggcgtggcctgggcgggactggggagtggcgagccctc

SEQ ID NO: 13 - DNA, 80 bp, Equine Infectious Anemia Virus
gctaggcaactaaaccgcaatatcctgtagttcctcatttgcgttccgcatttgtgacgtgttaagttcctgtttttaca

SEQ ID NO: 14 - DNA, 57 bp, Equine Infectious Anemia Virus
caatatcctgtagttcctcatttgcgttccgcatttgtgacgtgttaagttcctgtt

SEQ ID NO: 15 - DNA, 48 bp, Artificial Sequence
  A synthetic oligonucleotide
gtagttcctcaatatagttccgcatttgtgactagttaagttcctgtt

SEQ ID NO: 16 - DNA, 48 bp, Artificial Sequence
  A synthetic oligonucleotide
aacaggaacttaactagtcacaaatgcggaactatattgaggaactac

SEQ ID NO: 17 - DNA, 48 bp, Artificial Sequence; identical to SEQ ID NO: 16
  A synthetic oligonucleotide

SEQ ID NO: 18 - DNA, 84 bp, Equine Infectious Anemia Virus
tgttgctaggcaactaaaccgcaatatcctgtagttcctcatttgcgttccgcatttgtgacgtgttaagttcctgtttttaca

SEQ ID NO: 19 - DNA, 70 bp, Artificial Sequence
  A synthetic oligonucleotide
caatatcctgtagttcctcatttgcgttccgcatttgtgacgtgttaagttcctgttacggggagtggcg

SEQ ID NO: 20 - DNA, 70 bp, Artificial Sequence
  A synthetic oligonucleotide
tgttgctaggcaacaatatcctgtagttcctcatttgcgttccgcatttgtgacgtgttaagttcctgtt

SEQ ID NO: 21 - DNA, 83 bp, Artificial Sequence
  A synthetic oligonucleotide
tgttgctaggcaacaatatcctgtagttcctcatttgcgttccgcatttgtgacgtgttaagttcctgttacggggagtggcg

SEQ ID NO: 22 - DNA, 62 bp, Artificial Sequence
  A synthetic oligonucleotide
cgtagttcctcaatatagttccgcatttgtgactagttaagttcctgttacggggagtggcg

SEQ ID NO: 23 - DNA, 62 bp, Artificial Sequence
  A synthetic oligonucleotide
tgttgctaggcaagtagttcctcaatatagttccgcatttgtgactagttaagttcctgtta

SEQ ID NO: 24 - DNA, 74 bp, Artificial Sequence
  A synthetic oligonucleotide
tgttgctaggcaagtagttcctcaatatagttccgcatttgtgactagttaagttcctgttacggggagtggcg

SEQ ID NO: 25 - DNA, 70 bp, Human Immunodeficiency Virus
tggggactttccagggaggcgtggcctgggcgggactggggagtggcgagccctcagatgctgcatataa

SEQ ID NO: 26 - DNA, 71 bp, Equine Infectious Anemia Virus
caatatcctgtagttcctcatttgcgttccgcatttgtgacgtgttaagttcctgtttttacagtatataa

SEQ ID NO: 27 - DNA, 84 bp, Equine Infectious Anemia Virus
atatcctgtagttcctcatttgcgttccgcatttgtgacgtgttaagttcctgttacgcgtgagccctcagatgctgcatataa

SEQ ID NO: 28 - DNA, 76 bp, Artificial Sequence
  A synthetic oligonucleotide
tagttcctcaatatagttccgcatttgtgactagttaagttcctgttacgcgtgagccctcagatgctgcatataa

SEQ ID NO: 29 - DNA, 71 bp, Artificial Sequence
  A synthetic oligonucleotide
caatatcctgtagttcctcatttgcgttccgcatttgtgacgtgttaagttcctgttcctagggcatataa

SEQ ID NO: 30 - DNA, 62 bp, Artificial Sequence
  A synthetic oligonucleotide
gtagttcctcaatatagttccgcatttgtgactagttaagttcctgttcctagggcatataa

SEQ ID NO: 31 - DNA, 13 bp, Artificial Sequence
  A synthetic oligonucleotide
gttgctaggcaac

SEQ ID NO: 32 - DNA, 294 bp, Artificial Sequence
  A synthetic oligonucleotide
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrtnryynnrgnraynnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 33 - DNA, 14 bp, Artificial Sequence
  A synthetic oligonucleotide
atcgtcacggcgac

SEQ ID NO: 34 - DNA, 13 bp, Artificial Sequence
  A synthetic oligonucleotide
gttgcctagcaac

SEQ ID NO: 35 - DNA, 13 bp, Artificial Sequence
  A synthetic oligonucleotide
ttcccctagcaac

SEQ ID NO: 36 - DNA, 13 bp, Artificial Sequence
  A synthetic oligonucleotide
tttacccagcaac

SEQ ID NO: 37 - DNA, 13 bp, Artificial Sequence
  A synthetic oligonucleotide
ctttcctagcaac

SEQ ID NO: 38 - DNA, 13 bp, Artificial Sequence
  A synthetic oligonucleotide
tgcagctggcaac

SEQ ID NO: 39 - DNA, 13 bp, Artificial Sequence
  A synthetic oligonucleotide
tctgaccagcgac

SEQ ID NO: 40 - DNA, 13 bp, Artificial Sequence
  A synthetic oligonucleotide
tctgaccagcgac

SEQ ID NO: 41 - DNA, 14 bp, Artificial Sequence
  A synthetic oligonucleotide
gtccccatagcaac

SEQ ID NO: 42 - DNA, 13 bp, Artificial Sequence
  A synthetic oligonucleotide
ttcagctagtaac

SEQ ID NO: 43 - DNA, 13 bp, Artificial Sequence
  A synthetic oligonucleotide
gttaactagtaac

SEQ ID NO: 44 - DNA, 14 bp, Artificial Sequence
  A synthetic oligonucleotide
gttgttatagtaac

SEQ ID NO: 45 - DNA, 13 bp, Artificial Sequence
  A synthetic oligonucleotide
tttccttagtaac

SEQ ID NO: 46 - DNA, 14 bp, Artificial Sequence
  A synthetic oligonucleotide
gttactctggtgac

SEQ ID NO: 47 - DNA, 14 bp, Artificial Sequence
  A synthetic oligonucleotide
ccttttatggtaat

SEQ ID NO: 48 - DNA, 14 bp, Artificial Sequence
  A synthetic oligonucleotide
atcaccatggtaat

SEQ ID NO: 49 - DNA, 14 bp, Artificial Sequence
  A synthetic oligonucleotide
attaccatggtgat

SEQ ID NO: 50 - DNA, 13 bp, Artificial Sequence
  A synthetic oligonucleotide
attacctggtgat

SEQ ID NO: 51 - DNA, 14 bp, Artificial Sequence
  A synthetic oligonucleotide
gttgctatggccgc

SEQ ID NO: 52 - DNA, 14 bp, Artificial Sequence
  A synthetic oligonucleotide
atcgtcacggcgat

SEQ ID NO: 53 - DNA, 14 bp, Artificial Sequence
  A synthetic oligonucleotide
gttgtcatagtaat

SEQ ID NO: 54 - DNA, 13 bp, Artificial Sequence
  A synthetic oligonucleotide
gttgcccggcaac

SEQ ID NO: 55 - DNA, 13 bp, Artificial Sequence
  A synthetic oligonucleotide
gttgctaggtaac

SEQ ID NO: 56 - DNA, 13 bp, Artificial Sequence
  A synthetic oligonucleotide
gttgctaggtaac

SEQ ID NO: 57 - DNA, 14 bp, Artificial Sequence
  A synthetic oligonucleotide
gccgtcatggcgcc

SEQ ID NO: 58 - DNA, 20 bp, Artificial Sequence
  A synthetic primer
acgcgtcaatatcctgtagt

SEQ ID NO: 59 - DNA, 20 bp, Artificial Sequence
  A synthetic primer
acgcgtgtagttcctcaata

SEQ ID NO: 60 - DNA, 20 bp, Artificial Sequence
  A synthetic primer
acgcgtaacaggaacttaac

SEQ ID NO: 61 - DNA, 18 bp, Artificial Sequence
  A synthetic primer
acgcgtcccaagatttcc

SEQ ID NO: 62 - DNA, 22 bp, Artificial Sequence
  A synthetic primer
acgcgtctgatcctcttcttcc

SEQ ID NO: 63 - DNA, 21 bp, Artificial Sequence
  A synthetic primer
gaacaccagcttgttacaccc

SEQ ID NO: 64 - DNA, 21 bp, Artificial Sequence
  A synthetic primer
tctctctccttctagcctccg

SEQ ID NO: 65 - DNA, 36 bp, Artificial Sequence
  A synthetic primer
cctgttcctagggcatataagcagctcgtttttgcc

SEQ ID NO: 66 - DNA, 36 bp, Artificial Sequence
  A synthetic primer
ggacaaggatcccgtatattcgtcgacgaaaaacgc

SEQ ID NO: 67 - DNA, 37 bp, Artificial Sequence
  A synthetic primer
gaggatcagcctagggaggaggaagctcagatgctgc

SEQ ID NO: 68 - DNA, 37 bp, Artificial Sequence
  A synthetic primer
ctcctagtcggatccctcctccttcgagtctacgacg

SEQ ID NO: 69 - DNA, 46 bp, Artificial Sequence
  A synthetic primer
cctgttacggggagtggcgagccctaggatgctgcatataagcagc

SEQ ID NO: 70 - DNA, 46 bp, Artificial Sequence
  A synthetic primer
ggacaatgcccctcaccgctcgggatcctacgacgtatattcgtcg

SEQ ID NO: 71 - DNA, 46 bp, Artificial Sequence
  A synthetic primer
catcgagcttgctagctgttgctaggcaacaatatcctgtagttcc

SEQ ID NO: 72 - DNA, 46 bp, Artificial Sequence
  A synthetic primer
gtagctcgaacgatcgacaacgatccgttgttataggacatcaagg

SEQ ID NO: 73 - DNA, 39 bp, Artificial Sequence
  A synthetic primer
catcgagcttgctagctgttgctaggcaagtagttcctc

SEQ ID NO: 74 - DNA, 39 bp, Artificial Sequence
  A synthetic primer
gtagctcgaacgatcgacaacgatccgttcatcaaggag

SEQ ID NO: 75 - DNA, 32 bp, Artificial Sequence
  A synthetic oligonucleotide
tcctgtagttcctcaatatagttccgcatttg

SEQ ID NO: 76 - DNA, 23 bp, Artificial Sequence
  A synthetic oligonucleotide
tcatttgcgtgaagcatttctga

SEQ ID NO: 77 - DNA, 32 bp, Artificial Sequence
  A synthetic oligonucleotide
tagctcatgttgctaggcaactaaaccgcaat

SEQ ID NO: 78 - DNA, 14 bp, Artificial Sequence
  A synthetic oligonucleotide
rtnryyanrgnray

(19) United States
(12) Patent Application Publication
(10) Pub. No.: US 2006/0233757 A1
(43) Pub. Date: Oct. 19, 2006

(54) Vectors with viral insulators

(76) Inventor: Wendy Maury, Coralville, IA (US)

Correspondence Address: Schwegman, Lundberg, Woessner & Kluth, P.A., P.O. Box 2938, Minneapolis, MN, 55402, US

(21) Appl. No.: 11/215,829

(22) Filed: Aug. 29, 2005

Related U.S. Application Data
(60) Provisional application No. 60/604,898, filed on Aug. 27, 2004.

(51) Int. Cl. A61K 48/00; C12N 5/08; C12N 15/867

(52) U.S. Cl. 424/93.2; 435/366; 435/456; 435/325

(57) ABSTRACT

The invention provides a vector with an isolated viral insulator or variants thereof and uses therefor, e.g., gene therapy or transgenics.

### RELATED APPLICATION

This application claims priority under 35 U.S.C. 119(e) from U.S. Provisional Application Ser. No. 60/604,898 filed Aug. 27, 2004, which application is herein incorporated by reference.

### STATEMENT REGARDING FEDERALLY SPONSORED RESEARCH OR DEVELOPMENT

Work relating to this application was supported by a grant from the National Institutes of Health (NIH Grant No. CA72063). The government may have certain rights in the invention.

### BACKGROUND

Insulators represent a novel class of DNA sequences that constrain regulatory interactions within eukaryotic genomes. These elements restrict enhancer and silencer function and contribute to the generation of independent gene regulation within heterochromatic and euchromatic domains. Insulators are operationally defined by two functional properties. First, insulators protect gene expression from positive and negative chromatin effects caused when transgenes are integrated at random positions within a genome. Second, insulators block enhancer-activated transcription when the insulator is interposed between an enhancer and promoter, but not when the insulator is positioned upstream of the enhancer. Disruption of enhancer-activated transcription does not interfere with promoter function, since basal transcription of the target promoter is not affected (Geyer et al., 1992; Scott et al., 1995; Roseman et al., 1993). Similarly, enhancer blocking does not result in inactivation of enhancers (Scott et al., 1995; Cai et al., 1995). These data suggest that insulators interfere with mechanisms used in signaling between long-distance regulatory elements and promoters.

Insulators and related sequences have been identified in the genomes of the fruit fly, human, mouse, chicken, frog, sea urchin, budding yeast, and fission yeast. In many cases, an element has been classified as an insulator if it possesses at least one of the two functional properties. However, functional dissection of two insulators demonstrated that the enhancer-blocking and protection against position effects are conferred by distinct sequences, thereby uncoupling these activities (Litt et al., 2001; Saitoh et al., 2000; Willoughby et al., 2000). Thus, not all elements that possess one insulator property will possess both. Further, insulators may utilize more than one mechanism to impart regulatory autonomy. Geyer et al. (2002) proposed that the term insulator be reserved for sequences that both block enhancer-activated transcription and confer position-independent expression, and that sequences only capable of blocking enhancer function be called anti-enhancer elements.

Silencer elements are DNA binding sites for trans-acting factors that directly or indirectly reduce transcriptional initiation at promoters. Retrovirus vectors can have at least four separate silencer elements located in the LTRs and the adjacent primer binding site (PBS). Trans-acting factors can bind to silencers and can directly or indirectly recruit chromatin remodeling complexes. The CpG-rich silencer can be located in the LTR promoter, indicating that methylation-induced chromatin remodeling may not cause silencing mediated by the other three known silencers. Flanagan et al. (1989) identified a conserved upstream region in an LTR (now called the negative control region or NCR) that reduces virus expression, and identified mutations in the direct repeat (DR) of the LTR enhancer that increased expression in cells. Subsequent molecular characterization of the factors that bound to these regions identified several negative trans-acting factors. Specifically, the NCR binds the multi-functional transcriptional regulator YY-1 (Flanagan et al., 1992), and six distinct nuclear factors bind to each of the direct repeats (Speck et al., 1987). In addition, the embryonic LTR-binding protein (ELP), a homologue of Drosophila FTZ-F1 (Tsukiyama et al., 1992), was found to bind between the NCR and DR (Tsukiyama et al., 1989). It is interesting to note that save for ELP and factor A (which binds the PBS), all of the trans-acting factors that are known to bind the MoMLV LTRs are either ubiquitously expressed or not restricted to embryonic cells. As silencing still occurs when the ELP and factor A binding sites are mutated, the stem-cell specificity of retrovirus silencing remains unclear. In addition, it has been shown that no single silencer element is sufficient to induce complete silencing (Osborne et al., 1999), suggesting that silencer elements additively increase the probability of silencing.

The term anti-silencer, first introduced by Gilson and colleagues (Fourel et al., 1999), refers to an element that only has been demonstrated to block the action of transcriptional repressors or is only known to protect against position effects, as the majority of position effects appear to be negative (Stief et al., 1989; Pikaart et al., 1998). In some cases, anti-silencers have been called barriers, to reflect their distinction from insulators (Sun et al., 1999; Donze et al., 2002).

Several insulators and anti-enhancer elements prevent enhancer function when tested in different organisms (Saitoh et al., 2000; Akasaka et al., 1999; Zhong et al., 1997; Dunaway et al., 1997; Krebs et al., 1998; Chung et al., 1993). Similarly, many insulators and anti-silencers have the ability to block chromatin-mediated repression in different organisms (Willoughby et al., 2000; Pikaart et al., 1998; Chung et al., 1993; Antes et al., 2001; Chung et al., 1997; McKnight et al., 1992; Namciu et al., 1998; Phi-Van et al., 1996; Phi-Van et al., 1990; Takada et al., 2000; van der Vlag et al., 2000). Based on these observations, insulators and insulator-like sequences appear to be essential components of eukaryotic genomes that are required for establishment of appropriate levels of gene expression.

What is needed is an improved vector, e.g., a vector which once stably maintained in a cell, is less likely to be transcriptionally silenced.

### SUMMARY OF CERTAIN EMBODIMENTS OF THE INVENTION

It has been unexpectedly found that improved expression from genes delivered by viral vectors, e.g., long term expression, can be improved by strategic insertion of at least one insulator.

Accordingly, the present invention provides a vector imcluding a chimeric recombinant DNA molecule including one or more insulators, one or more transcriptional regulatory control sequences, and an open reading frame for a gene product, wherein at least one insulator is at least 10 nucleotides but no more than about 150 nucleotides in length and is 5′ to at least one transcriptional regulatory control sequence which is operably linked to the open reading frame and/or optionally 3′ to the open reading frame. Also provided is a plasmid including a vector of the invention, and a host cell such as a mammalian host cell including a plasmid and/or vector of the invention. One embodiment provides a plurality of insulators 3′ to the open reading frame. Another embodiment provides a plurality of insulators 5′ to the open reading frame. Another embodiment provides a poly(A) site 3′ to the open reading frame.

The present invention also provides a vector including one or more copies of an insulator wherein at least one insulator is at least 10 nucleotides but no more than about 150 nucleotides in length.

Also provided is a method to alter the expression of an open reading frame in a cell, including contacting a cell with a vector and/or plasmid of the invention so as to yield a cell which stably expresses the open reading frame at a level which is different than the level of expression in a corresponding cell having a corresponding vector or corresponding plasmid which lacks the one or more insulators.

Also provided is a method to inhibit or treat a condition associated with aberrant expression of an endogenous gene product, including: contacting a mammal at risk of or having the condition, with an effective amount of a vector and/or plasmid and/or host cell of the invention, which vector, plasmid or host cell contains an open reading frame encoding at least a portion of a functional gene product, the expression of which in the mammal inhibits or treats at least one symptom of the condition.

Also provided is a method to express an open reading frame in a vector which is stably associated with a mammalian cell including stably introducing to the mammalian cell a vector and/or plasmid of the invention so as to yield a stably genetically transformed mammalian cell which expresses the open reading frame.

Also provided is a method of preparing a vector including: a) providing at least one selected isolated insulator, and an expression cassette including one or more transcriptional regulatory control sequences operably linked to an open reading frame or one or more isolated transcriptional regulator control sequences and an isolated open reading frame, wherein at least one insulator is at least 10 nucleotides but no more than about 150 nucleotides in length; and b) introducing the at least one isolated insulator, and expression cassette or isolate transcriptional regulatory control sequence and isolated open reading frame into a vector to yield a vector having a chimeric DNA molecule including the at least one isolated insulator 5′ and/or 3′ to the transcriptional regulatory control sequence operably linked to the open reading frame. The present invention also provides vectors prepared by the methods of the invention. One embodiment provides a selected insulator of formula I: X₂RT^{m}YRYYX₁^{m}YRG^{m}YRAYX₃ (SEQ ID NO:32), wherein X₁ is A or is absent, R is a purine, Y is a pyrimidine, ^{m}Y is thymidine or 5-methylcytosine, and X₂ and X₃ are individually absent, a nucleotide, or a nucleotide sequence of 2 or more and up to about 140 nucleotides. In one embodiment, the vector is a viral vector. In another embodiment, at least one transcriptional regulatory control sequence is a promoter or an enhancer. In one embodiment, at least one transcriptional regulatory control sequence and at least one insulator are heterologous. One embodiment provides that the insulator contains from 13 to about 50 nucleotides.

Also provided is a transgenic animal, e.g., which is not a human, including at least one of a vector, plasmid, or host cell of the invention.

The present invention is directed to an isolated nucleic acid sequence which includes at least a 10 nucleotide sequence, e.g., a 13 nucleotide sequence, e.g., one identified as a methylation dependent protein binding (MDBP) site, a fragment and/or a variant thereof, that has the characteristics of a transcriptional insulator (boundary element) and, in the context of a viral vector, optionally enhances viral replication. An insulator of the invention can thus protect the expression of a gene against the influence of the integration site (position effect), and/or acts as a positional enhancer blocker in the sense that an enhancer that is 3′ to the insulator can enhance transcription but an enhancer that is 5′ to the insulator has reduced or no activity on expression cassettes 3′ to the insulator. The nucleotide sequence and variants thereof are found in a number of viral enhancers (such as hepatitis B virus and cytomegalovirus) as well as in the enhancer/promoter region of some mammalian genes including α-galactosidase A, tumor growth factor-1β and collagen α2(I). The role of the sequence in mammalian genes seems to be primarily as a negative transcriptional element, however, in equine infectious anemia virus (EIAV), the sequence stimulates virus replication. Thus, while this sequence may not serve as an insulator in its native context in mammalian promoters, it may serve as one in a vector, e.g., a viral vector. Interestingly, methylation of the MDBP site in mammalian promoters facilitates the binding of RFX protein members to the sequence, but not in viruses.

In contrast to other insulators, the present insulator sequences are very short and are the first such elements described in a retrovirus. As discussed above, one problem with the use of integrating viruses in gene therapy is long term transgene expression from the integrated virus. In general, the transgene is silenced over time by upstream and downstream effects. The introduction of one or more insulators of the invention to a vector for stable gene delivery may block genomic silencing effects and thus allow the transgene longer and stronger expression within the genome. Since insulators can separate transcriptional domains and prevent upstream (5′) sequences from affecting downstream (3′) events, vectors with such an insulator have value in gene delivery systems, e.g., transgenes, and in gene therapy. In one embodiment, the insulator is flanked by other sequences (heterologous or chimeric sequences) at the 5′ and/or 3′ end, and in one embodiment is a viral sequence or a variant thereof.

Thus, insertion of a one or more insulators of the invention, such as one having formula I: X₂RT^{m}YRYYX₁^{m}YRG^{m}YRAYX₃ (SEQ ID NO:32), wherein X₁ is A or is absent, R is a purine, Y is a pyrimidine, ^{m}Y is thymidine or 5-methylcytosine, and X₂ and X₃ are individually absent, a nucleotide, or a nucleotide sequence of 2 or more and up to about 140 nucleotides, in vectors such as plasmids and viruses that are used to stably deliver transgenes into cells is beneficial for long term expression of an open reading frame of interest in those vectors. The insulator or a tandem array of one or more insulators are inserted in a 5′ location and/or a 3′ location relative to the open reading frame of interest, e.g., an expression cassette including a promoter, and optionally an enhancer, operably linked to the open reading frame (transgene). Insertion of insulators of the invention in a transgene containing vector insulates the transgene and its enhancer/promoter sequence from surrounding chromosomal sequences once the vector is stably maintained in, e.g., integrated into the chromosome of, a cell and thus reduces or eliminates undesirable upstream and downstream effects, e.g., silencing. For instance, in one embodiment, an insulator of the invention is placed immediately upstream from a heterologous enhancer/promoter linked to an open reading frame of interest within a plasmid, and/or 3′ to the open reading frame or 3′ to a polyadenylation site which flanks the open reading frame. For example, an insulator can be linked to a therapeutic gene to prevent the negative effects on the expression of the open reading frame for therapeutic gene product caused by the site of integration. Thus, wild-type (functional) versions of genes can be introduced into cells in vitro or in vivo to alter the phenotype of those cells or a mammal having those cells.

In one embodiment, the invention provides for vectors useful to transfer an open reading frame into cells, such as viral vectors, e.g., retroviral, adeno-associated virus, or lentiviral vectors such as equine infectious anemia virus (EIAV)-derived vectors. Generally, the insulator is placed near the ends of the transgene and for viral vectors near the ends of the viral genome, for instance, for retroviral or lentiviral vectors, within the 5′ and/or 3′ LTR at a position that is 5′ to the promoter in the LTR, forming a chimeric LTR, i.e., a LTR sequence not found in wild-type retroviruses or lentiviruses, which optionally has heterologous sequences, i.e., those derived from two different sources. The insulator of the invention is one which is generally 150 nucleotides in length or less, e.g., 125, 100, 75, 60, 50, 40, 30, 20 or 15 nucleotides in length, but greater than 9 nucleotides in length, and may include contiguous sequences found in formula I. The insulator of the invention is introduced via recombinant DNA methodologies and/or homologous recombination to a nucleic acid molecule (vector, e.g., plasmid), and is positioned upstream of transcriptional regulatory control sequences selected to control expression of a linked open reading frame. In one embodiment, the insulator is internal to the ends of an LTR in a retroviral or lentiviral vector and at least 5′ and optionally 3′ to an expression cassette juxtaposed between the LTRs. In one embodiment, a retroviral or lentiviral vector includes an insulator within a 5′ LTR and/or 3′ LTR, a packaging signal, and optionally a multiple cloning site for introduction of an open reading frame, and optionally a heterologous promoter and/or enhancer. Nevertheless, the insulator(s) may be employed in any vector intended to stably deliver a gene to a cell. In one embodiment, the insulator(s) may be employed in any vector intended to transiently deliver a gene to a cell.

The invention also provides a method of producing a transgene containing vector, such as a lentiviral or retroviral vector, for stable expression of the transgene. Also provided is a method of introducing an open reading frame of interest to a host cell, which includes introducing to the host cell a vector containing one or more insulators of the invention. The host cell may be any eukaryotic cell and optionally is a mammalian cell, e.g., a rodent, bovine, equine, swine, caprine, ovine, feline, canine, non-human primate, or human cell.

In one embodiment, the insulator of the invention is used to insulate or buffer the expression of an open reading frame from the action of an adjacent regulatory element, such as an enhancer or silencer, after the open reading frame is inserted into the DNA of a cell. The method includes providing an insulator upstream of a promoter for the open reading frame (for retroviruses or lentiviral, such a promoter may be in the 3′ LTR of a retroviral or lentiviral vector) prior to introducing the open reading frame into the cell. In one embodiment, the open reading frame is flanked at both ends by one or more insulator sequences. In one embodiment, the open reading frame is inserted into a polylinker site of a vector, wherein the polylinker of the vector is flanked by one or more insulators. In addition, the vector can be further provided with a selectable marker gene. Once the open reading frame has been inserted into the polylinker of the vector, the vector is introduced to a cell in vitro or in vivo using techniques available to those skilled in the art. Expression of the integrated open reading frame is then insulated from any cis-acting DNA regulatory sequences present in the chromatin of the cell.

The insulator containing vectors of the present invention thus allow for a more precise control of the expression of open reading frame after introduction of the vector to cells due to the reduced impact/elimination of position effects. In particular, the present invention provides a nucleic acid vector and method for insulating the expression of an open reading frame in transgenic animals such that the introduced expression cassette is protected and stably expressed in the tissues of the transgenic animal or its offspring. For example, the introduced expression cassette is expressed even if the DNA of the vector integrates into areas of silent or active chromatin in the genomic DNA of the host animal.

In one embodiment, a vector is provided that includes one or more insulators, a tissue specific promoter and/or enhancer, and a polylinker, wherein the insulator is 5′ to the promoter and/or 3′ to a site for insertion of an open reading frame. Use of the present insulator with a tissue-specific promoter and/or enhancer may ensure strict tissue-specific expression of a linked gene.

In one embodiment a kit is provided. The kit may include vectors including the insulator of the invention optionally linked to a polylinker or open reading frame packaged in a container, e.g., vials, tubes, microtiter well plates, and the like. Other reagents can be included in separate containers and provided with the kit, e.g., positive control samples, negative control samples, buffers, cell culture media, etc.

### BRIEF DESCRIPTION OF THE FIGURES

FIG. 1. EMSA binding results using nuclear extracts from primary macrophages and the fibroblast cell line, Cf₂Th cells. Transcription factor motifs are shown. All double stranded oligonucleotides were tested between 24 and 30 nucleotides long (SEQ ID NOs:2-10) and were derived from the MA-1 sequence (SEQ ID NO: 1). All binding was specific for the oligonucleotide as demonstrated by competition studies (data not shown). Competition studies were also performed using mutated oligonucleotides to determine which sites within an oligonucleotide were binding to the NE (data not shown). NE from primary macrophages had specific binding of oligonucleotides containing either ets or octamer sites. Interestingly, while specific binding of macrophage NE to octamer sites was apparent, octamer sites are not present in macrophage tropic strains of EIAV. Specific binding of Cf₂Th cell NE occurred in oligonucleotides containing a MDBP, PEA-2, octamer or CREB site. In addition, specific binding with Cf₂Th NE can be observed with nucleotides −90 to −80.

FIG. 2. Site directed mutations within the enhancer region of MA-1 (SEQ ID NO:1 1). Transient transfections were performed in Cf₂Th cells. Relative activity of the mutant LTR is shown as percent of wild LTR/CAT activity (basal MA-1 LTR/CAT activity=1). Values in parenthesis are the fold activation over basal levels in the presence of EIAV Tat.

FIG. 3. Chimeric LTR enhancer regions. HIV enhancer/promoter proximal region with 2 NF-κB and three Sp1 motifs (SEQ ID NO:12). This region has been deleted and replaced with a MluI restriction site in the pCMlu construct that was used to generate chimeric LTRs. A prototypic EIAV enhancer is shown with transcription factor binding motifs identified above the sequence (SEQ ID NO:13). In addition to the core PU.1 motif that is highlighted, three of the four nucleotides immediately downstream of the core site influence PU.1 binding (Maury, 1994). In the MT LTR (SEQ ID NO: 14), three additional transcription factor binding motifs, Lvb, Oct, and CRE, are also present. 3PU1 (SEQ ID NO:15), 3PU1R (SEQ ID NO:16), and 3PU13X (SEQ ID NO:17) enhancer sequences are derived from a modified MT enhancer that had deleted all known transcription factor binding motifs except the PU.1 binding sites. 3PU1R within in the context of the LTR is in the reverse orientation. 3PU13X is a concatemer of three sense PU1 enhancers. All amplified enhancer sequences were inserted into the MluI site present in the pCMlu LTR.

FIG. 4. PU.1 expression is needed for optimal chimeric LTR/reporter activity (A) Basal activity of the LTR. (B) Tat-transactivated activity of the LTR. HEK 293 cells were transfected with the chimeric LTR/CAT constructs and PU.1 or empty vector in the absence or presence of a HIV Tat expression vector. Percentage of acetylation values represent the mean values with standard error. All experiments were performed in triplicate and repeated at least two independent times. Open box represents tranfections containing the empty vector; shaded box represents transfections containing PU.1 expressing plasmid. Note difference in γ-axis between A and B.

FIG. 5. Endogenous transcription factors present in DH82 macrophages are sufficient for chimeric LTR/CAT activity, but not in Jurkat T cells. (A) An electrophoretic mobility shift assay with oligonucleotide 124 from the EIAV LTR was used as a ³²P-labeled probe. This probe encompasses −87 to −56 of the EIAV enhancer region that contains the 5′ and middle PU.1 sites and has been shown to specifically bind to PU.1 in primary macrophage nuclear extracts (Maury, 1994). A 200-fold excess of oligonucleotide competitors was used to demonstrate specificity of binding. 136GAA is an EIAV-based oligonucleotide that has the core PU.1 motif mutated from TTCC to TGAA and is not able to bind to PU.1 (Maury, 1994). Oligonucleotide 91 is composed of EIAV LTR nucleotides −118 to −88 that does not contain a PU.1 site. The arrow highlights the slowest migrating specific complex. (B) Basal LTR activity in a CAT reporter gene assay. (C) Tat-transactivated LTR activity in a CAT reporter gene assay. Percentage of acetylation values represent the mean values with the standard error. All experiments were performed in triplicate and repeated a minimum of two independent times. Note difference in γ-axis in B and C.

FIG. 6. Addition of MDBP and/or Sp1 transcription factor binding motifs to the chimeric LTR enhancer sequences enhanced transcriptional activity in macrophages, but not T cells. (A) The pMT and p3PU1 chimeric LTR/CATs were modified by addition of a MDBP and/or a Sp1 motif singly or in combination (SEQ ID NOs: 18-24). The Sp1 motif (represented as S in the nomenclature in the clone name) was placed at the 3′ end of the enhancer, whereas the MDBP motif (represented as M in the nomenclature in the clone name) was placed at the 5′ end of the enhancer. (B) Basal levels of LTR activity in DH82 macrophages. (C) Tat-transactivated levels of LTR activity in Jurkat T cells. Percentage of acetylation values represent mean plus standard error. All experiments were done in triplicate and repeated a minimum of two independent times.

FIG. 7. Spacing between enhancer sequences and the promoter is important for optimal expression of the HIV LTR. (A) Nucleotide spacing between the 3′ transcription factor motif and TATA box of WT and chimeric LTRs was altered to reflect wild type spacing found the EIAV LTR (SEQ ID NOs:25-30). (B) Tat-transactivated expression of chimeric LTR/CAT constructs in DH82 cells. (C) Tat-transactivated expression of chimeric LTR/CAT constructs in Jurkat T cells. Percentage of acetylation values represent mean plus standard error. All experiments were done in triplicate and repeated a minimum of two independent times.

FIG. 8. Viruses containing chimeric LTRs replicate in a macrophage-specific manner. (A) Monocyte-derived macrophage infections using the dual tropic HIV molecular clone p256. (B) Jurkat T cell infections using the dual tropic HIV molecular clone p256. Cpm values represent the mean of two independent infections. (C) MDM (open symbols) and CD4⁺ T cell (filled symbols) infections using VSVG pseudotyped viruses. (D) HeLa37 and HeLa37 clone 7 cell infections using the dual tropic HIV molecular clone support higher p256. Serial dilutions of 48 or 72 hours virus stocks from HEK293 cell transfections were tested on either HeLa 37 cells or HeLa37 clone 7 cells for entry and HIV antigen expression at 40 hours post-infection. Values shown represent the number of HIV antigen expressing cells/ml of viral stock.

FIG. 9. Amplified sequences from HIV constructs containing chimeric LTR sequences are readily detectable in Jurkat T cells at day 6 post-infection, indicating that these constructs are not blocked at entry. (A) Schematic of the HIV LTR showing positions of BglII restriction sites in relation to primer start sites and transcription start. (B) Ethidium bromide stained agarose gel of amplified proviral LTR/gag sequences. Genomic DNA was amplified using primers HIV-264 in U3 and HIV+335C′ in the 5′ untranslated region (UTR) of gag from 6 day infected Jurkat T cells. Amplified product was digested with BglII that cuts twice at nucleotides +19 and +208 generating fragments of 283, 189, and 127 bp. The largest fragment varied depending on the size of enhancer present. Undigested amplified product was run in parallel. Arrow on right denotes PCR product; red arrow is nonspecific PCR product; arrow heads indicate restriction enzyme products.

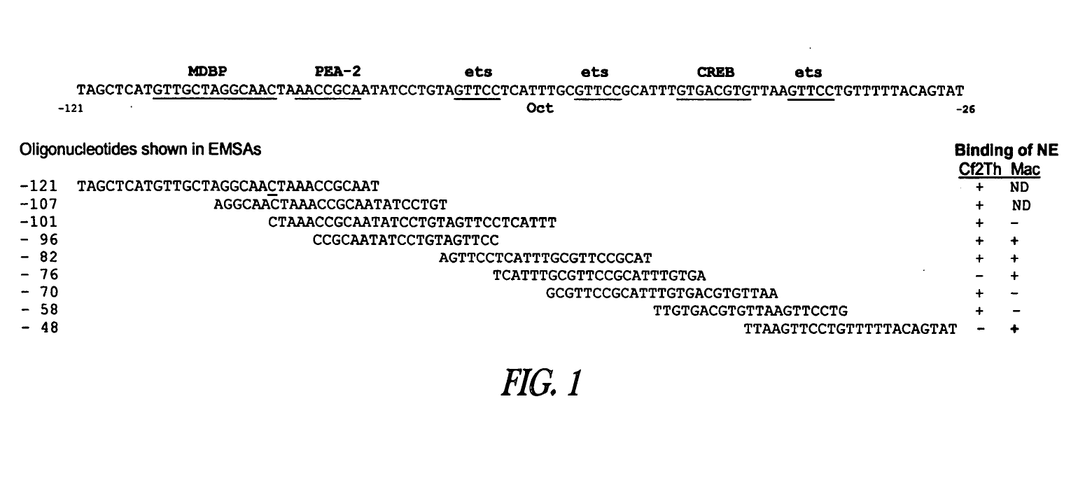

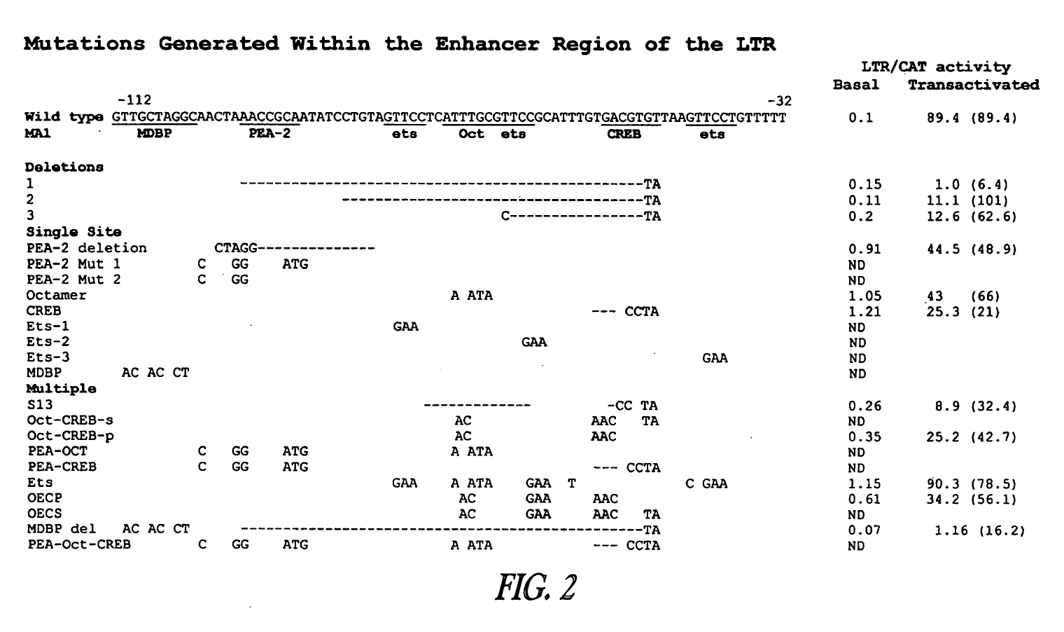

### DETAILED DESCRIPTION OF THE INVENTION

It has been unexpectedly found that expression of genes delivered by viral vectors, e.g., long term expression, can be improved by strategic insertion of at least one insulator.

Accordingly, the present invention provides a vector including a chimeric recombinant DNA molecule including one or more insulators, one or more transcriptional regulatory control sequences, and an open reading frame for a gene product, wherein at least one insulator is at least 10 nucleotides but no more than about 150 nucleotides in length and is 5′ to at least one transcriptional regulatory control sequence which is operably linked to the open reading frame and/or optionally 3′ to the open reading frame.

In some embodiments of the invention, the transcriptional regulatory control sequence which is operably linked to the open reading frame is a promoter. In some embodiments of the invention, the promoter is a viral promoter. In some embodiments of the invention, the promoter is a cellular promoter. In some embodiments of the invention, the promoter is heterologous to at least one insulator.

In some embodiments of the invention, at least one insulator is 5′ to transcriptional regulatory control sequences which include an enhancer and a promoter. In some embodiments of the invention, the enhancer is heterologous to the promoter.

In some embodiments of the invention, the vector is a viral vector. In some embodiments of the invention, the vector is a retroviral vector, a lentiviral vector, or an adeno-associated virus vector.

In some embodiments of the invention, one or more insulators are 3′ to the open reading frame. In some embodiments of the invention, a plurality of insulators are 3′ to the open reading frame. In some embodiments of the invention, a plurality of insulators are 5′ to the open reading frame.

In some embodiments of the invention, the vector further includs a poly(A) site 3′ to the open reading frame. In some embodiments of the invention, at least one insulator is introduced into the 3′ LTR of a retroviral or lentiviral vector. In some embodiments of the invention, at least one insulator is introduced into the 5′ ITR and optionally the 3′ ITR of an adeno-associated viral vector.

In some embodiments of the invention, at least one insulator includes GTTGCTAGGCAAC (SEQ ID NO:31). In some embodiments of the invention, at least one insulator includes a sequence of formula I: X₂RT^{m}YRYYX₁^{m}YRG^{m}YRAYX₃ (SEQ ID NO:32), wherein X₁ is A or is absent, R is a purine, Y is a pyrimidine, ^{m}Y is thymidine or 5-methylcytosine, and X₂ and X₃ are individually absent, a nucleotide, or a nucleotide sequence of 2 or more and up to about 140 nucleotides. In some embodiments of the invention, the open reading frame encodes a therapeutic gene product. In some embodiments of the invention, the open reading frame encodes a prophylactic gene product. In some embodiments of the invention, the open reading frame encodes a catalytic RNA.

The present invention also provides a plasmid including a vector of the invention, and a host cell including the vector and/or plasmid of the invention. In some embodiments of the invention, the host cell is a mammalian host cell. In some embodiments of the invention, the vector is stably integrated into the genome of the mammalian cell. In some embodiments of the invention, the vector is an adenovirus, lentivirus, adeno-associated virus (AAV), polyomavirus, herpes simplex virus, or retrovirus vector.

The present invention also provides a vector including one or more copies of an insulator wherein at least one insulator is at least 10 nucleotides but no more than about 150 nucleotides in length.

The present invention further provides a method to alter the expression of an open reading frame in a cell, including contacting a cell with a vector and/or plasmid of the invention so as to yield a cell which stably expresses the open reading frame at a level which is different than the level of expression in a corresponding cell having a corresponding vector or corresponding plasmid which lacks the one or more insulators.

The present invention also provides a method to inhibit or treat a condition associated with aberrant expression of an endogenous gene product, including: contacting a mammal at risk of or having the condition, with an effective amount of a vector, plasmid, and/or host cell of the invention which vector, plasmid or host cell contains an open reading frame encoding at least a portion of a functional gene product, the expression of which in the mammal inhibits or treats at least one symptom of the condition. In some embodiments of the invention, prior to contacting, the mammal lacks expression of the endogenous gene product. In some embodiments of the invention, prior to contacting, the mammal overexpresses the endogenous gene product. In some embodiments of the invention, prior to contacting, the mammal has reduced expression of the endogenous gene product.

The present invention also provides a method to express an open reading frame in a vector which is stably associated with a mammalian cell including: stably introducing to the mammalian cell a vector or plasmid of the invention so as to yield a stably genetically transformed mammalian cell which expresses the open reading frame. In some embodiments of the invention, the gene product is a reporter protein. In some embodiments of the invention, the gene product is a prophylactic or therapeutic gene product.

The present invention also provides a method of preparing a vector including: a) providing at least one selected isolated insulator, and an expression cassette including one or more transcriptional regulatory control sequences operably linked to an open reading frame or one or more isolated transcriptional regulator control sequences and an isolated open reading frame, wherein at least one insulator is at least 10 nucleotides but no more than about 150 nucleotides in length; and b) introducing the at least one isolated insulator, and expression cassette or isolate transcriptional regulatory control sequence and isolated open reading frame into a vector to yield a vector having a chimeric DNA molecule including the at least one isolated insulator 5′ and/or 3′ to the transcriptional regulatory control sequence operably linked to the open reading frame. In some embodiments of the invention, the selected insulator has formula I: X₂RT^{m}YRYYX₁^{m}YRG^{m}YRAYX₃ (SEQ ID NO:32), wherein X₁ is A or is absent, R is a purine, Y is a pyrimidine, ^{m}Y is thymidine or 5-methylcytosine, and X₂ and X₃ are individually absent, a nucleotide, or a nucleotide sequence of 2 or more and up to about 140 nucleotides. In some embodiments of the invention, the vector is a viral vector. In some embodiments of the invention, at least one transcriptional regulatory control sequence is a promoter. In some embodiments of the invention, at least one transcriptional regulatory control sequence is an enhancer. In some embodiments of the invention, at least one transcriptional regulatory control sequence and at least one insulator are heterologous. In some embodiments of the invention, the insulator contains from 13 to about 50 nucleotides.

The present invention also provides a vector prepared by the method of the invention. The present invention also provides a plasmid prepared by the method of the invention. The present invention also provides a host cell prepared by the method of the invention.

The present invention also provides a transgenic animal including at least one of a vector, plasmid, or host cell of the invention. In some embodiments of the invention, the transgenic animal is not a human.

Definitions

An insulator is a segment of DNA that serves to isolate a gene by blocking interactions, e.g., between enhancers on one side of the insulating sequence from the promoters of neighboring genes. Evidence presented herein indicates that improved expression from genes delivered by viral vectors, e.g., long term expression, can be improved by strategic insertion of at least one insulator. For purposes of the present invention, the term is to be understood in a broad functional sense. The defining characteristics of an insulating sequence within the meaning of the invention is its ability to insulate or protect a defined open reading frame which is operably linked to a transcriptional regulatory control sequence from the influence of an upstream element when located between the upstream element and the regulatory sequence, e.g., a promoter linked to an open reading frame to be insulated. Insulators useful in the present invention may be of any origin, including both plant, fungi, yeast and animal species, including insects (e.g., Drosophila), mammals (e.g., rat, mouse, dog, cat), birds (e.g., chicken, turkey), and the like, as well as insulators of viral origin. Where two or more insulators are employed, they may be the same or different. The length of the insulator is not critical, but may generally be from about 10 bases up to about 150 nucleotides, e.g., 20, 30, or more nucleotides in length. The insulating sequence or a variant thereof may then be inserted into a vector such as a viral vector at a suitable position, i.e., the resulting vector is chimeric.

A “vector” or “construct” (sometimes referred to as gene delivery or gene transfer “vehicle”) refers to a macromolecule or complex of molecules including a polynucleotide to be delivered to a host cell, either in vitro or in vivo. The polynucleotide to be delivered may include a sequence of interest for gene therapy. Vectors include, for example, transposons and other site-specific mobile elements, viral vectors, e.g., adenovirus, adeno-associated virus (AAV), poxvirus, papillomavirus, lentivirus, herpesvirus, foamivirus and retrovirus vectors, and including pseudotyped viruses, liposomes and other lipid-containing complexes, and other macromolecular complexes capable of mediating delivery of a polynucleotide to a host cell, e.g., DNA coated gold particles, polymer-DNA complexes, liposome-DNA complexes, liposome-polymer-DNA complexes, virus-polymer-DNA complexes, e.g., adenovirus-polylysine-DNA complexes, and antibody-DNA complexes. Vectors can also include other components or functionalities that further modulate gene delivery and/or gene expression, or that otherwise provide beneficial properties to the cells to which the vectors will be introduced. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

A “recombinant viral vector” refers to a viral vector including one or more heterologous genes or sequences. Since many viral vectors exhibit size constraints associated with packaging, the heterologous genes or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying genes necessary for replication and/or encapsidation). Modified viral vectors in which a polynucleotide to be delivered is carried on the outside of the viral particle have also been described (see, e.g., Curiel et al. 1991).

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, iontophoresis, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

By “transgene” is meant any piece of a nucleic acid molecule (for example, DNA) which is inserted by artifice into a cell either transiently or permanently (stably), and becomes part of the organism if integrated into the genome or maintained extrachromosomally. Such a transgene may include a gene which is partly or entirely heterologous (i.e., foreign) to the transgenic organism, or may represent a gene homologous to an endogenous gene of the organism.

By “transgenic cell” is meant a cell containing a transgene. For example, a stem cell transformed with a vector containing an expression cassette can be used to produce a population of cells having altered phenotypic characteristics.

The term “wild-type” refers to a gene or gene product that has the characteristics of that gene or gene product when isolated from a naturally occurring source. For example, a wild type gene can refer to a gene that produces a protein that has normal function. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “transduction” denotes the delivery of a polynucleotide to a recipient cell either in vivo or in vitro, via a viral vector and preferably via a replication-defective viral vector.

The term “chimeric” as it relates to nucleic acid sequences such as insulators and control sequences or control sequences and open reading frames, denotes sequences that are not normally joined, for example, operably linked, together. In some embodiments, the “chimeric” sequence has insertion(s), deletion(s), substitution(s) and/or is not normally associated with a particular cell. Thus, a “chimeric” region of a nucleic acid construct or a vector is a segment of nucleic acid within or attached to another nucleic acid molecule that is not found in association with the other molecule in nature. For example, a “chimeric” sequence may be an insulator that is located within a sequence of a vector in a position not seen in nature. For example, a chimeric region of a nucleic acid construct could include a coding sequence flanked by sequences not found in association with the coding sequence in nature, i.e., a heterologous promoter. As used herein, “heterologous” sequences denote sequences from two different sources, e.g., two different species. Another example of a chimeric coding sequence is a construct where the coding sequence itself is not found in nature, e.g., the coding sequence includes synthetic sequences having codons different from the native gene. A cell transformed with a construct having sequences which are not normally present in the cell would be considered heterologous for purposes of this invention. Thus, in some embodiments of the invention, the vectors of the invention are “chimeric” and/or “heterologous” vectors.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

A “gene,” “polynucleotide,” “coding region,” “segment,” “fragment” or “sequence” which “encodes” a particular gene product, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. Thus, a gene includes a polynucleotide which may include a full-length open reading frame which encodes a gene product (sense orientation) or a portion thereof (sense orientation) which encodes a gene product with substantially the same activity as the gene product encoded by the full-length open reading frame, the complement of the polynucleotide, e.g., the complement of the full-length open reading frame (antisense orientation) and optionally linked 5′ and/or 3′ noncoding sequence(s) or a portion thereof, e.g., an oligonucleotide, which is useful to inhibit transcription, stability or translation of a corresponding mRNA. A transcription termination sequence will usually be located 3′ to the gene sequence.

An “oligonucleotide” includes at least 7 nucleotides, preferably 15, and more preferably 20 or more sequential nucleotides, up to 100 nucleotides, either RNA or DNA, which correspond to the complement of the non-coding strand, or of the coding strand, of a selected mRNA, or which hybridize to the mRNA or DNA encoding the mRNA and remain stably bound under moderately stringent or highly stringent conditions, as defined by methods well known to the art, e.g., in Sambrook et al., A Laboratory Manual, Cold Spring Harbor Press (2001).

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter region” is used herein in its ordinary sense to refer to a nucleotide region including a DNA regulatory sequence, wherein the regulatory sequence is derived from a gene which is capable of binding RNA polymerase and initiating transcription of a downstream (3′ direction) coding sequence. Thus, a “promoter,” refers to a polynucleotide sequence that controls transcription of a gene or coding sequence to which it is operably linked. A large number of promoters, including constitutive, inducible and repressible promoters, from a variety of different sources, are available to the art.

By “enhancer element” is meant a nucleic acid sequence that, when positioned proximate to a promoter, confers increased transcription activity relative to the transcription activity resulting from the promoter in the absence of the enhancer domain. Hence, an “enhancer” includes a polynucleotide sequence that enhances transcription of a gene or coding sequence to which it is operably linked. A large number of enhancers, from a variety of different sources are well known in the art. A number of polynucleotides which have promoter sequences (such as the commonly-used CMV promoter) also have enhancer sequences.

“Operably linked” refers to a juxtaposition, wherein the components so described are in a relationship permitting them to function in their intended manner. By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. A promoter is operably linked to a coding sequence if the promoter controls transcription of the coding sequence. Although an operably linked promoter is generally located upstream of the coding sequence, it is not necessarily contiguous with it. An enhancer is operably linked to a coding sequence if the enhancer increases transcription of the coding sequence. Operably linked enhancers can be located upstream, within or downstream of coding sequences. A polyadenylation sequence is operably linked to a coding sequence if it is located at the downstream end of the coding sequence such that transcription proceeds through the coding sequence into the polyadenylation sequence.

“Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. Thus, a signal or targeting peptide sequence is operably linked to another protein if the resulting fusion is secreted from a cell as a result of the presence of a secretory signal peptide or into an organelle as a result of the presence of an organelle targeting peptide.

“Homology” refers to the percent of identity between two polynucleotides or two polypeptides. The correspondence between one sequence and to another can be determined by techniques available to the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single strand-specific nuclease(s), and size determination of the digested fragments. Two DNA, or two polypeptide, sequences are “substantially homologous” to each other when at least about 80%, preferably at least about 90%, and most preferably at least about 95% of the nucleotides, or amino acids, respectively match over a defined length of the molecules, as determined using the methods above.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like. An “animal” includes vertebrates such as mammals, avians, amphibians, reptiles and aquatic organisms including fish.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

By “expression construct” or “expression cassette” is meant a nucleic acid molecule that is capable of directing transcription. An expression construct includes, at the least, a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means, or in relation to a cell refers to a cell which was isolated and subsequently introduced to other cells or to an organism by artificial or natural means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “isolated” when used in relation to a nucleic acid, peptide, polypeptide or virus refers to a nucleic acid sequence, peptide, polypeptide or virus that is identified and separated from at least one contaminant nucleic acid, polypeptide, virus or other biological component with which it is ordinarily associated in its natural source. Isolated nucleic acid, peptide, polypeptide or virus is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

The term “recombinant DNA molecule” as used herein refers to a DNA molecule that is included of segments of DNA joined together by means of molecular biological techniques.

The term “recombinant protein” or “recombinant polypeptide” as used herein refers to a protein molecule that is expressed from a recombinant DNA molecule.

The terms “peptide”, “polypeptide” and “protein” are used interchangeably herein unless otherwise distinguished to refer to polymers of amino acids of any length. These terms also include proteins that are post-translationally modified through reactions that include glycosylation, acetylation and/or phosphorylation.

The term “sequence homology” refers to the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

The term “selectively hybridize” means to detectably and specifically bind. Polynucleotides, oligonucleotides and fragments of the invention selectively hybridize to nucleic acid strands under hybridization and wash conditions that minimize appreciable amounts of detectable binding to nonspecific nucleic acids. High stringency conditions can be used to achieve selective hybridization conditions as available to the art and discussed herein. Generally, the nucleic acid sequence homology between the polynucleotides, oligonucleotides, and fragments of the invention and a nucleic acid sequence of interest is at least 65%, and more typically with preferably increasing homologies of at least about 70%, about 90%, about 95%, about 98%, and 100%.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of more than 5 (in standard deviation units) using the program ALIGN with the mutation data matrix and a gap penalty of 6 or greater. See Dayhoff, 1972. The two sequences or parts thereof are more preferably homologous if their amino acids are greater than or equal to 50% identical when optimally aligned using the ALIGN program.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may include a complete cDNA or gene sequence. Generally, a reference sequence is at least 20 nucleotides in length, frequently at least 25 nucleotides in length, and often at least 50 nucleotides in length. Since two polynucleotides may each (1) include a sequence (i.e., a portion of the complete polynucleotide sequence) that is similar between the two polynucleotides, and (2) may further include a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may include additions or deletions (i.e., gaps) of 20 percent or less as compared to the reference sequence (which does not include additions or deletions) for optimal alignment of the two sequences. Optimal alignment of sequences for aligning a comparison window may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide includes a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least about 80 percent sequence identity, preferably at least about 90 percent sequence identity, more preferably at least about 95 percent sequence identity, and most preferably at least about 99 percent sequence identity.

As used herein, “substantially pure” means an object species is the predominant species present (i.e., on a molar basis it is more abundant than any other individual species in the composition), and preferably a substantially purified fraction is a composition wherein the object species includes at least about 50 percent (on a molar basis) of all macromolecular species present. Generally, a substantially pure composition will include more than about 80 percent of all macromolecular species present in the composition, more preferably more than about 85%, about 90%, about 95%, and about 99%. Most preferably, the object species is purified to essential homogeneity (contaminant species cannot be detected in the composition by conventional detection methods) wherein the composition consists essentially of a single macromolecular species.

As used herein, the term “ameliorating” means that the clinical signs or the symptoms of the pathologic condition are lessened. The skilled clinician will recognize that amelioration of the severity of a pathologic condition can be identified, for example, by performing clinical tests specific for determining the progress of the particular disease. For example, where the pathologic condition is a cancer, amelioration of the severity of the disease can be determined using imaging techniques that allow monitoring of the size or growth rate of a tumor. Where the pathologic condition is associated with damage to a particular tissue or organ, the clinician will recognize that amelioration of the pathologic condition can be determined, for example, by performing enzymatic assays relevant to the state of a tissue or organ affected with the pathologic condition.

As used herein, the term “inducible,” when used in reference to a transcriptional regulatory control sequence, means a nucleotide sequence that, when present in a cell exposed to an inducing agent, effects an increased level of transcription of an operatively linked expressible polynucleotide as compared to the level of transcription, if any, in the absence of an inducing agent. The term “inducing agent” is used to refer to a chemical, biological or physical agent that effects transcription from an inducible transcriptional regulatory control sequence. In response to exposure to an inducing agent, transcription from the element generally is initiated de novo or is increased above a basal or constitutive level of expression. Such induction can be identified, for example, by detecting an increased level of a reporter polypeptide encoded by an expressible polynucleotide that is operatively linked to the transcriptional regulatory element. An inducing agent can be, for example, a stress condition to which a cell is exposed, for example, a heat or cold shock, a toxic agent such as a heavy metal ion, or a lack of a nutrient, hormone, growth factor, or the like; or can be exposure to a molecule that affects the growth or differentiation state of a cell such as a hormone, a cytokine, or a growth factor.

A “variant” nucleotide sequence includes a deletion of one or more nucleotides, a substitution of one or more nucleotides, and/or an insertion of one or more nucleotides, relative to a reference nucleotide sequence. The variant may be derived from a naturally occurring sequence by appropriately modifying the sequence to add, remove, and/or to modify nucleotides including codons for one or more amino acids at one or more sites of the C-terminus, N-terminus, and/or within the naturally occurring or reference sequence. It is understood that such variants having added, substituted and/or additional nucleotides retain one or more characterizing portions of the reference nucleotide sequence. A derivative may be prepared using standard techniques well-known to those of ordinary skill in the art.

By “expression” is meant production of the encoded product, preferably by transcription and/or translation of the desired gene or nucleic acid sequence.

By “desired gene” is meant to refer to any gene or nucleic acid sequence encoding a gene product. Examples of desired gene product include prophylactic gene products and therapeutic gene products (e.g., those capable of at least partially reducing or preventing one or more symptoms of a disease). Gene products include proteins, polypeptides, or RNA, e.g., siRNA, antisense RNA or a ribozyme.

The term “persistent expression” as used herein refers to introduction of genes into the cell together with genetic elements which enable episomal (i.e., extrachromosomal) replication. This can lead to apparently stable transformation of the cell without the integration of the novel genetic material into the chromosome of the host cell.

“Stable expression” as used herein relates to the integration of genetic material into chromosomes of the targeted cell where it becomes a permanent component of the genetic material in that cell. Gene expression after stable integration can permanently alter the characteristics of the cell and its progeny arising by replication leading to stable transformation.

The term “plasmid” as used herein generally refers to a circular duplex of DNA which can replicate independently of chromosomal DNA. The plasmid does not necessarily replicate. The term “plasmid” includes genetic elements arranged such that an inserted coding sequence can be transcribed in eukaryotic cells. The plasmid may include a sequence from a viral nucleic acid. Preferably a plasmid is a closed circular DNA molecule.

By “delivery” or “delivering” is meant transportation of nucleic acid molecules to desired cells or any cells. The nucleic acid molecules will be delivered to multiple cell lines, including the desired target. Delivery results in the nucleic acid molecules coming in contact with the cell surface, cell membrane, cell endosome, within the cell membrane, nucleus or within the nucleus, or any other desired area of the cell from which transfection can occur within a variety of cells.

The term “organism” as used herein refers to common usage by one of ordinary skill in the art. The organism can include; microorganisms, such as yeast or bacteria, plants, birds, reptiles, fish or mammals. The organism can be a companion animal or a domestic animal. Preferably the organism is a mammal and is therefore any warm blooded organism. More preferably the mammal is a human.

The term “domestic animal” as used herein refers to those animals traditionally considered domesticated, where animals such as those considered “companion animals” are included along with animals such as a pig, chicken, duck, cow, goat, sheep, dog, cat, horse, bovine and the like.

As used herein, “cell,” “cell line,” and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid or nucleic acid-protein complex into the subject organism, e.g., patient.

Identification of Insulators

To test whether a sequence can function as an insulator, the sequence can be cloned into a vector which includes, 5′ to 3′, a enhancer, the sequence to be tested, a promoter such as an inducible or constituitive promoter (e.g., an HSP70 promoter), and a reporter gene (e.g., gus, luciferase and the like). Expression of the reporter gene should be constitutively suppressed in the absence of the appropriate signal (e.g., temperature) for the inducible promoter. If presence of the sequence to be tested in the vector decreases the expression of the reporter gene, then the sequence may function as an insulator.

In general, insulating sequences do not have a direct effect on transcription in transient transfection assays. Instead these elements impact transcription in the setting of chromatin by either altering the chromatin structure and/or preventing upstream or downstream elements from affecting a transcriptional promoter element. One assay that the art worker can use to define an insulator is to place the potential element into an upstream region of a cassette that can be expressed transiently in the unintegrated state or be expressed stably when it becomes integrated into chromatin. An insulator would have no effect on expression in the unintegrated state, but would impact the expression of the plasmid in the integrated state.

Exemplary Insulator Sequences

An insulator is a sequence that serves to isolate a gene by blocking interactions, e.g., between enhancers on one side of the insulating sequence from the promoters of neighboring genes. Evidence presented herein indicates that expression from genes delivered by viral vectors, e.g., long term expression, can be improved by strategic insertion of at least one insulator. For purposes of the present invention, the term is to be understood in a broad functional sense. A defining characteristics of an insulating sequence within the meaning of the invention is its ability to insulate or protect a defined open reading frame which is operably linked to a transcriptional regulatory control sequence from the influence of an upstream element when located between the upstream element and the regulatory sequence, e.g., a promoter linked to an open reading frame to be insulated.

Exemplary insulator sequences include sequences that include: sequences of formula I including RT^{m}YRYYA^{m}YRG^{m}YRAY (SEQ ID NO:78), ATMGTCAMGGMGAC (SEQ ID NO:33), GTTGCCTAGCAAC (SEQ ID NO:34), TTCCCCTAGCAAC (SEQ ID NO:35), TTTACCCAGCAAC (SEQ ID NO:36), CTTTCCTAGCAAC (SEQ ID NO:37), TGCAGCTGGCAAC (SEQ ID NO:38), TCTGACCAGCGAC (SEQ ID NO:39), TCTGACCAGMGAC (SEQ ID NO:40), GTCCCCATAGCAAC (SEQ ID NO:41), TTCAGCTAGTAAC (SEQ ID NO:42), GTTAACTAGTAAC (SEQ ID NO:43), GTTGTTATAGTAAC (SEQ ID NO:44), TTTCCTTAG TAAC (SEQ ID NO:45), GTTACTCTGGTGAC (SEQ ID NO:46), CCTTTTATGGTAAT (SEQ ID NO:47), ATCACCATGGTAAT (SEQ ID NO:48), ATTACCATGGTGAT (SEQ ID NO:49), ATTACCTGGTGAT (SEQ ID NO:50), GTTGCTATGGCCGC (SEQ ID NO:51), ATMGTCAMGGMGAT (SEQ ID NO:52), GTTGTCATAGTAAT (SEQ ID NO:53), GTTGCCCGGCAAC (SEQ ID NO:54), GTTGCTAGGTAAC (SEQ ID NO:55), GTTGCTAGGTAAC (SEQ ID NO:56), GCMGTCATGGMGCC (SEQ ID NO:57) and GTTGCTAGGCAAC (SEQ ID NO:31), (R, purine; Y, pyrimidine; ^{m}Y, either T or 5-methylcytosine; M, 5-methylcytosine).

Preparation of Expression Cassettes

To prepare expression cassettes for transformation, the recombinant DNA sequence or segment may be circular or linear, double-stranded or single-stranded. A DNA sequence which encodes an RNA sequence that is substantially complementary to a mRNA sequence encoding a gene product of interest is typically a “sense” DNA sequence cloned into a cassette in the opposite orientation (i.e., 3′ to 5′ rather than 5′ to 3′). Generally, the DNA sequence or segment is in the form of chimeric DNA, such as plasmid DNA, that can also contain coding regions flanked by control sequences which promote the expression of the DNA in a cell. As used herein, “chimeric” means that a vector includes DNA from at least two different species, or includes DNA from the same species, which is linked or associated in a manner which does not occur in the “native” or wild-type of the species.

Aside from DNA sequences that serve as transcription units, or portions thereof, a portion of the DNA may be untranscribed, serving a regulatory or a structural function. For example, the DNA may itself include a promoter that is active in eukaryotic cells, e.g., mammalian cells, or in certain cell types, or may utilize a promoter already present in the genome that is the transformation target of the lymphotropic virus. Such promoters include the CMV promoter, as well as the SV40 late promoter and retroviral LTRs (long terminal repeat elements), e.g., the MMTV, RSV, MLV or HIV LTR, although many other promoter elements well known to the art may be employed in the practice of the invention.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

The recombinant DNA to be introduced into the cells may contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, puro, hyg, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Exemplary reporter genes include the chloramphenicol acetyl transferase gene (cat) from Tn9 of E. coli, the beta-glucuronidase gene (gus) of the uidA locus of E. coli, the green, red, or blue fluorescent protein gene, and the luciferase gene. Expression of the reporter gene is assayed at a suitable time after the DNA has been introduced into the recipient cells.

The general methods for constructing recombinant DNA which can transform target cells are available to those skilled in the art, and the same compositions and methods of construction may be utilized to produce the DNA useful herein. For example, Sambrook et al. (2001) provides suitable methods of construction.

The recombinant DNA can be readily introduced into the host cells, e.g., mammalian, bacterial, yeast or insect cells, or prokaryotic cells, by transfection with an expression vector including the recombinant DNA by any procedure useful for the introduction into a particular cell, e.g., physical or biological methods, to yield a transformed (transgenic) cell having the recombinant DNA so that the DNA sequence of interest is expressed by the host cell. In one embodiment, at least one of the recombinant DNAs which is introduced to a cell is maintained extrachromosomally. In one embodiment, one recombinant DNA is maintained extrachromosomally while another is stably integrated into its genome.

Physical methods to introduce a recombinant DNA into a host cell include calcium-mediated methods, lipofection, particle bombardment, microinjection, electroporation, and the like. Biological methods to introduce the DNA of interest into a host cell include the use of DNA and RNA viral vectors. Viral vectors, e.g., retroviral or lentiviral vectors, have become a widely used method for inserting genes into eukaryotic, such as mammalian, e.g., human, cells. Other viral vectors useful to introduce genes into cells can be derived from poxviruses, e.g., vaccinia viruses, herpes viruses, adenoviruses, adeno-associated viruses, baculoviruses, and the like.

“Transfected,” “transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

To confirm the presence of the recombinant DNA sequence in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular gene product, e.g., by immunological means (ELISAs and Western blots) or by other molecular assays.

To detect and quantitate RNA produced from introduced recombinant DNA segments, RT-PCR may be employed. In this application of PCR, RNA is reverse transcribed into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and demonstrate the presence or absence of an RNA species.

While Southern blotting and PCR may be used to detect the recombinant DNA segment in question, they do not provide information as to whether the recombinant DNA segment is being expressed. Expression may be evaluated by specifically identifying the peptide products of the introduced DNA sequences or evaluating the phenotypic changes brought about by the expression of the introduced DNA segment in the host cell.

Vectors

The present invention relates to a recombinant DNA molecule including 5′ to 3′, an optional enhancer, a promoter, an open reading frame of interest, including a start codon, a stop codon, or a ribosome binding site, an optional 3′ noncoding region, e.g., a polyA site, and, depending on the vector, an insulator 5′ to the optional enhancer, an insulator 3′ to the optional poly(A) site, or both. The open reading frame of interest encodes an RNA molecule that has an inherent activity, for example, an antisense molecule, a ribozyme, or a triplexing agent, one that can be used as a screenable molecule that, for example, by detecting its presence using a specific hybridization reaction, a reporter or selectable marker, which facilitates identification or isolation of a cell containing the polypeptide, a polypeptide such as one useful in gene therapy.

An expression cassette can be monocistronic, encoding only a single RNA molecule or single polypeptide, or can be polycistronic, encoding two or more RNA molecules, polypeptides, or a combination thereof. A polycistronic polynucleotide provides the advantage that one or more reporter molecules can be expressed in a cell, thus providing a means to identify or isolate cells containing the vector including the polynucleotide, and that an additional one or more polypeptides of interest can be expressed in the cell. The promoter may be the naturally occurring promoter for the operably linked open reading frame or heterologous to the open reading frame. If desired, a non-coding region 3′ to the sequence encoding a desired gene product may be included for transcriptional termination, such as termination and polyadenylation. Thus, the 3′ noncoding region may be homologous or heterologous to the open reading frame. The present invention also relates to a cell or organism that contains a recombinant DNA molecule and is capable of expressing the open reading frame. A cell is said to be “altered to express a desired gene product” when the cell, through genetic manipulation, is made to produce a gene product which it normally does not produce or which the cell normally produces but is produced at different levels. One skilled in the art can readily adapt procedures for introducing and expressing either genomic, cDNA, or synthetic sequences into either eukaryotic or prokaryotic cells.

A nucleic acid molecule, such as DNA, is capable of expressing a gene product if it contains nucleotide sequences which contain transcriptional and translational regulatory information and such sequences are “operably linked” to nucleotide sequences which encode the gene product. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but will in general include a promoter region and may include 5′-non-coding sequences involved with initiation of transcription and translation, such as the TATA box, capping sequence, CAAT sequence, and the like.

The present invention encompasses the expression of a desired open reading frame in eukaryotic cells. However, a vector of the invention may be propagated in prokaryotic cells, e.g., E. coli. In prokaryotic systems, plasmid vectors that contain replication sites and control sequences derived from a species compatible with the host may be used. Examples of suitable plasmid vectors may include pBR322, pUC118, pUC119 and the like; suitable phage or bacteriophage vectors may include λ-gt10, λ-gt11 and the like; and suitable virus vectors may include pMAM-neo, pKRC and the like. Preferably, the selected vector of the present invention has the capacity to replicate in the selected host cell. Recognized prokaryotic hosts include bacteria such a E. coli and those such as Bacillus, Streptomyces, Pseudomonas, Salmonella, Serratia, and the like. The prokaryotic host must be compatible with the replicon and control sequences in the expression plasmid.

Eukaryotic hosts include, for example, yeast, fungi, insect cells, mammalian cells either in vivo, or in tissue culture. Many types of cells and cell lines (e.g., primary cell lines or established cell lines) and tissues are capable of being stably transfected by or receiving the vectors of the invention. Examples of cells that may be used include, but are not limited to, stem cells, B lymphocytes, T lymphocytes, macrophages, other white blood lymphocytes (e.g., myelocytes, macrophages, monocytes), immune system cells of different developmental stages, erythroid lineage cells, pancreatic cells, lung cells, muscle cells, liver cells, fat cells, neuronal cells, glial cells, other brain cells, transformed cells of various cell lineages corresponding to normal cell counterparts (e.g., K562, HEL, HL60, and MEL cells), and established or otherwise transformed cell lines derived from all of the foregoing. In addition, the vectors of the present invention may be transferred by various means directly into tissues, where they would stably integrate into the cells including the tissues. Further, the vectors containing the insulator elements of the invention can be introduced into primary cells at various stages of development, including the embryonic and fetal stages, so as to effect gene therapy at early stages of development.

A wide variety of transcriptional and translational regulatory sequences may be employed, depending upon the nature of the host cell. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated.

Examples of eukaryotic promoters suitable for use in the invention include, but are not limited to, the thymidine kinase (TK) promoter, the alpha globin, beta globin, and gamma globin promoters, the human or mouse metallothionein promoter, the SV40 promoter, retroviral promoters, cytomegalovirus (CMV) promoter, and the like. Accordingly, promoters may be homologous or heterologous. Suitable promoters may be inducible, allowing induction of the expression of a gene upon addition of the appropriate inducer, or they may be non-inducible.

Further, a variety of eukaryotic enhancer elements may be used in the constructs of the invention. Like the promoters, the enhancer elements may be homologous or heterologous. Examples of suitable enhancers include, but are not limited to, erythroid-specific enhancers, the immunoglobulin enhancer, virus-specific enhancers, e.g. SV40 enhancers, or viral LTRs, pancreatic-specific enhancers, muscle-specific enhancers, fat cell-specific enhancers, liver specific enhancers, and neuron-specific enhancers.

A desired nucleic acid molecule and an operably linked promoter flanked by at least one insulator may be introduced into a recipient prokaryotic or eukaryotic cell either as a linear DNA (or RNA) molecule, a closed covalent circular molecule (a plasmid), or via a delivery vehicle, e.g., a virus.

Preferably a vector of the invention is one which integrates or is capable of integrating the desired open reading frame into the host cell chromosome. Cells which have stably integrated the introduced DNA into their chromosomes may be selected or detected by also introducing one or more selectable or detectable markers which allow for selection of host cells which contain the vector. The selectable marker gene sequence can either be directly linked to the DNA gene sequences to be expressed, or introduced into the same cell by co-transfection. Additional elements may also be needed for optimal synthesis, including splice signals, as well as enhancers, and/or termination signals.

In one embodiment, a recombinant DNA molecule of the invention is incorporated into a plasmid or viral vector capable of autonomous replication in the recipient host. Any of a wide variety of vectors may be employed for this purpose.

Preferred prokaryotic vectors include plasmids such as those capable of replication in E. coli (such as, for example, pBR322, ColE1, pSC101, pACYC 184, pVX). Such plasmids are, for example, disclosed by Sambrook et al. (2001). Bacillus plasmids include pC194, pC221, pT127, and the like. Such plasmids are disclosed by Gryczan (1982). Suitable Streptomyces plasmids include plJ101 (Kendall et al., 1987), and Streptomyces bacteriophages such as fC31 (Chater et al., 1986). Pseudomonas plasmids are reviewed in John et al. (1986)), and Izaki (1978). Preferred eukaryotic plasmids include, for example, BPV, vaccinia, SV40, 2-micron circle, and the like, or their derivatives. Such plasmids are available to the art (Botstein et al., 1982; Broach, 1981; Broach, 1982; Bollon et al., 1980; Maniatis, 1980).

Exemplary Vector and Method

The vectors as described herein may be used in gene transfer and gene therapy methods to allow the protected expression of one or more given genes that are stably transfected into the cellular DNA. The vectors of the invention would not only insulate an expression cassette from the influences of DNA surrounding the site of integration, but may also prevent activation of the transcription of genes that are harmful or detrimental to the cell.

The vectors of the invention are capable of being introduced into a variety of cell and tissue types. In addition, the insulator element itself may not be cell or tissue specific, and so can act as a part of the vectors of the invention to insulate gene expression in the absence of strict cell or tissue specificity. The vectors can be designed to contain the appropriate regulatory sequences and optionally all of the necessary DNA elements for integration of the vector and/or the appropriate components thereof and expression of a gene of interest in a given cell type.

For assembly of the vector, the insulator element for ligation can be positioned in accordance with the desired use of the vector. Thus, as disclosed above, at least one vector may be positioned 5′ and/or 3′ to a transcription unit so as to obtain optimal insulation of the gene or genes desired to be transcribed. The insulator element can be obtained from natural sources or by synthetic means. For example, the insulator element can be excised from genomic DNA clones of eukaryotes, including chickens, mice, and humans, and the like, or synthesized by conventional techniques of DNA synthesis, and then ligated with segments of DNA including the transcription unit.

Those skilled in the art will appreciate that a variety of enhancers, promoters, and genes are suitable for use in the vectors of the invention, and that the vectors will contain the necessary start, termination, and control sequences for proper transcription and processing of the gene of interest when the construct is introduced into a mammalian or a higher eukaryotic cell. The constructs may be introduced into cells by a variety of gene transfer methods available to those skilled in the art, for example, gene transfection, microinjection, electroporation, and infection. In addition, it is envisioned that the invention can encompass all or a portion of a viral sequence-containing vector, such as those described in U.S. Pat. No. 5,112,767, for targeted delivery of genes to specific tissues. It is preferred that the constructs of the invention integrate stably into the genome of specific and targeted cell types.

Further, the DNA construct including the insulator element, promoter and transcription unit may be inserted into or assembled within a vector such as a plasmid, cosmid or virus for incorporation into the host cell of interest. The vectors may contain a bacterial origin of replication so that they can be amplified in a bacterial host. The vectors may also contain, in addition to a selectable marker for selection of transfected cells, as in the exemplary constructs, another expressible and selectable gene of interest.

Vectors can be constructed which have the insulator element in appropriate relation to an insertion region for receiving DNA encoding a gene product thereof. The insertion region can contain at least one restriction enzyme recognition site.

A particularly useful vector for gene therapy is the retroviral vector. A recombinant retroviral vector may contain the following parts: an intact 5′ LTR from an appropriate retrovirus, followed by DNA containing the retroviral packaging signal sequence; a insulator element, a promoter and a transcription unit containing a gene to be introduced into a specific cell for gene therapy; optionally a selectable gene as described below; and a 3′ LTR which optionally contains a insulator element in the U3 region. The selectable gene may or may not have a 5′ promoter that is active in the packaging cell line, as well as in the transfected cell. In one embodiment, one or more retroviral splice sites or start codons between the site of transcription initiation and the start codon for the open reading frame of interest are mutated.

The recombinant retroviral vector DNA can be transfected into the packaging cell line which is capable of producing high titer stocks of helper-free recombinant retroviruses. After transfection, the packaging cell line may be selected for resistance to G418, present at appropriate concentration in the growth medium.

Examples of transfectable reporter genes that can be used in the present invention include those genes whose function is desired or needed to be expressed in vivo or in vitro in a given cell or tissue type. Genes having significance for genetic or acquired disorders are particularly appropriate for use in the constructs and methods of the invention. Genes that may be insulated from cis-acting regulatory sequences by the insulator elements of the present invention may be selected from, but are not limited to, both structural and non-structural genes, or subunits thereof, such as those which encode proteins and glycoproteins (e.g., factors (e.g., growth factors), cytokines, lymphokines), enzymes (e.g., enzymes in biosynthetic pathways), hormones, which perform normal physiological, biochemical, and biosynthetic functions in cells and tissues. Other useable genes are selectable antibiotic resistance genes (e.g., the neomycin phosphotransferase gene (Neo®), the methotrexate-resistant dihydrofolate reductase (dhfr) gene) or drug resistance genes (e.g., the multi-drug resistance (MDR) genes), and the like. Further, the genes may encode a precursor of a particular protein, or the like, which is modified intracellularly after translation to yield the molecule of interest.

Gene Therapy Vectors

Gene therapy vectors include, for example, viral vectors, liposomes and other lipid-containing complexes, and other macromolecular complexes capable of mediating delivery of a gene to a host cell. Vectors can also include other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Gene therapy vectors within the scope of the invention include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary gene therapy vectors are described below. Gene therapy vectors may be administered via any route including, but not limited to, intramuscular, buccal, rectal, intravenous or intracoronary administration, and transfer to cells may be enhanced using electroporation and/or iontophoresis.

Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range (Schnepp et al., 2002).

Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells, e.g., after direction injection or perfusion. Adenoviral vectors have been shown to result in transient expression of therapeutic genes in vivo, peaking at 7 days and lasting approximately 4 weeks. In addition, adenoviral vectors can be produced at very high titers, allowing efficient gene transfer with small volumes of virus.

Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans.

Herpesvirus/Amplicon

Herpes simplex virus 1 (HSV-1) has a number of important characteristics that make it an important gene delivery vector in vivo. There are two types of HSV-1-based vectors: 1) those produced by inserting the exogenous genes into a backbone virus genome, and 2) HSV amplicon virions that are produced by inserting the exogenous gene into an amplicon plasmid that is subsequently replicated and then packaged into virion particles. HSV-1 can infect a wide variety of cells, both dividing and nondividing, but has obviously strong tropism towards nerve cells. It has a very large genome size and can accommodate very large transgenes (>35 kb).

Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. For example, expression of secreted angiogenesis factors after muscle injection of plasmid DNA, despite relatively low levels of focal transgene expression, has demonstrated significant biologic effects in animal models and appears promising clinically (Isner, 2002). Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

Synthetic Oligonucleotides

Antisense oligonucleotides are short (approximately 10 to 30 nucleotides in length), often chemically synthesized DNA molecules that are designed to be complementary to the coding sequence of an RNA of interest. These agents may enter cells by diffusion or liposome-mediated transfer and possess relatively high transduction efficiency. These agents are useful to reduce or ablate the expression of a targeted gene while unmodified oligonucleotides have a short half-life in vivo, modified bases, sugars or phosphate groups can increase the half-life of oligonucleotides. For unmodified nucleotides, the efficacy of using such sequences is increased by linking the antisense segment with a specific promoter of interest, e.g., in an adenoviral construct. In one embodiment, electroporation and/or liposomes are employed to deliver plasmid vectors. Synthetic oligonucleotides may be delivered to cells as part of a macromolecular complex, e.g., a liposome, and delivery may be enhanced using techniques such as electroporation.

Recombinant cells or vectors may be administered intravenously, transvenously, or by any other convenient route, and delivered by a needle, catheter, e.g., a catheter which includes an injection needle or infusion port, or other suitable device.

Targeted Vectors

The present invention contemplates the use of cell targeting not only by delivery of the transgene or recombinant cell into the coronary artery, for example, but also by use of targeted vector constructs having features that tend to target gene delivery and/or gene expression to particular host cells or host cell types (such as the myocardium). Such targeted vector constructs would thus include targeted delivery vectors and/or targeted vectors, as described herein. Restricting delivery and/or expression can be beneficial as a means of further focusing the potential effects of gene therapy. The potential usefulness of further restricting delivery/expression depends in large part on the type of vector being used and the method and place of introduction of such vector. For instance, delivery of viral vectors via intracoronary injection to the myocardium has been observed to provide, in itself, highly targeted gene delivery. In addition, using vectors that do not result in transgene integration into a replicon of the host cell (such as adenovirus and numerous other vectors), cardiac myocytes are expected to exhibit relatively long transgene expression since the cells do not undergo rapid turnover. In contrast, expression in more rapidly dividing cells would tend to be decreased by cell division and turnover. However, other means of limiting delivery and/or expression can also be employed, in addition to or in place of the illustrated delivery method, as described herein.

Targeted delivery vectors include, for example, vectors (such as viruses, non-viral protein-based vectors and lipid-based vectors) having surface components (such as a member of a ligand-receptor pair, the other half of which is found on a host cell to be targeted) or other features that mediate preferential binding and/or gene delivery to particular host cells or host cell types. As is known in the art, a number of vectors of both viral and non-viral origin have inherent properties facilitating such preferential binding and/or have been modified to effect preferential targeting (see, e.g., Miller, et al., 1995; Chonn et al., 1995; Schofield et al., 1995; Schreier, 1994; Ledley, 1995; WO 95/34647; WO 95/28494; and WO 96/00295).

Targeted vectors include vectors (such as viruses, non-viral protein-based vectors and lipid-based vectors) in which delivery results in transgene expression that is relatively limited to particular host cells or host cell types. For example, transgenes can be operably linked to heterologous tissue-specific enhancers or promoters thereby restricting expression to cells in that particular tissue.

Dosages and Dosage Forms

The amount of vectors, plasmids and/or cells, e.g., gene therapy vector(s), e.g., those which are present in a recombinant cell or in acellular form, administered to achieve a particular outcome will vary depending on various factors including, but not limited to, the gene and promoter chosen, the condition, patient specific parameters, e.g., height, weight and age, and whether prevention or treatment is to be achieved. The gene therapy vector of the invention is amenable to chronic use for prophylactic purposes. For gene therapy, the vectors of the invention may be introduced to any mammal, e.g., a mammal having symptoms of a genetically-based disorder, an acquired disorder or an infectious disease which is amenable to gene-based therapy, including but not limited to bovine, ovine, equine, caprine, canine, feline, and porcine, as well as primates, particularly humans. The introduction into the mammalian host may be by any of several routes, including intravenous or intraperitoneal injection, intratracheally, intrathecally, parenterally, intraarticularly, intranasally, intramuscularly, topical, transdermal, application to any mucous membrane surface, corneal instillation, and the like. For certain vectors, certain routes of administration may be preferred. For instance, viruses, e.g., pseudotyped virus, and DNA- or virus-liposome, e.g., HVJ-liposome, may be intravenously administered.

Vectors of the invention may conveniently be provided in the form of formulations suitable for administration, e.g., into the blood stream (e.g., in an intracoronary artery). A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. Vectors of the present invention should preferably be formulated in solution at neutral pH, for example, about pH 6.5 to about pH 8.5, more preferably from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, more preferably from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions. If desired, solutions of the above compositions can also be prepared to enhance shelf life and stability. Therapeutically useful compositions of the invention can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors can be provided in a dosage form containing an amount of a vector effective in one or multiple doses. For viral vectors, the effective dose may be in the range of at least about 10⁷ viral particles, preferably about 10⁹ viral particles, and more preferably about 10¹¹ viral particles. The number of viral particles may, but preferably does not exceed 10¹⁴. As noted, the exact dose to be administered is determined by the attending clinician, but is preferably in 1 ml phosphate buffered saline. For delivery of recombinant cells, the number of cells to be administered will be an amount which results in a beneficial effect to the recipient. For example, from 10² to 10¹⁰, e.g., from 10³ to 10⁹, 10⁴ to 10⁸, or 10⁵ to 10⁷, cells can be administered. For delivery of plasmid DNA alone, or plasmid DNA in a complex with other macromolecules, the amount to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered.

By way of illustration, liposomes and other lipid-containing gene delivery complexes can be used for delivery. The principles of the preparation and use of such complexes for delivery have been described in the art (see, e.g., Ledley, 1995; Miller et al., 1995; Chonn et al., 1995; Schofield et al., 1995; Brigham et al., 1993).

Administration of the gene therapy vector in accordance with the present invention may be continuous or intermittent, depending, for example, upon the recipient's physiological condition, whether the purpose of the administration is therapeutic or prophylactic, and other factors known to skilled practitioners. The administration of the gene therapy vector may be essentially continuous over a preselected period of time or may be in a series of spaced doses. Both local and systemic administration is contemplated.

One or more suitable unit dosage forms including the gene therapy vector, e.g., a recombinant cell including the vector, which may optionally be formulated for sustained release, can be administered by a variety of routes including oral, or parenteral, including by rectal, buccal, vaginal and sublingual, transdermal, subcutaneous, intravenous, intramuscular, intraperitoneal, intrathoracic, intrapulmonary and intranasal routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

Pharmaceutical formulations containing the gene therapy vector can be prepared by procedures available to the art using well known and readily available ingredients. For example, the agent can be formulated with common excipients, diluents, or carriers, and formed into tablets, capsules, suspensions, powders, and the like. The vectors of the invention can also be formulated as elixirs or solutions for convenient oral administration or as solutions appropriate for parenteral administration, for instance by intramuscular, subcutaneous or intravenous routes.

The pharmaceutical formulations of the vectors can also take the form of an aqueous or anhydrous solution or dispersion, or alternatively the form of an emulsion or suspension.

Thus, the vector may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are available to the art. It is possible, for example, to prepare solutions using one or more organic solvent(s) that is/are acceptable from the physiological standpoint.

For administration to the upper (nasal) or lower respiratory tract by inhalation, the vector is conveniently delivered from an insufflator, nebulizer or a pressurized pack or other convenient means of delivering an aerosol spray. Pressurized packs may include a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the vector may be administered via nose drops, a liquid spray, such as via a plastic bottle atomizer or metered-dose inhaler. Typical of atomizers are the Mistometer (Wintrop) and the Medihaler (Riker).

The local delivery of the vectors can also be by a variety of techniques which administer the vector at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the vectors may be formulated as is known in the art for direct application to a target area. Conventional forms for this purpose include wound dressings, coated bandages or other polymer coverings, ointments, creams, lotions, pastes, jellies, sprays, and aerosols, as well as in toothpaste and mouthwash, or by other suitable forms. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

When desired, the above-described formulations can be adapted to give sustained release of the active ingredient employed, e.g., by combination with certain hydrophilic polymer matrices, e.g., including natural gels, synthetic polymer gels or mixtures thereof.

Drops, such as eye drops or nose drops, may be formulated with an aqueous or non-aqueous base also including one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The vector may further be formulated for topical administration in the mouth or throat. For example, the active ingredients may be formulated as a lozenge further including a flavored base, usually sucrose and acacia or tragacanth; pastilles including the composition in an inert base such as gelatin and glycerin or sucrose and acacia; mouthwashes including the composition of the present invention in a suitable liquid carrier; and pastes and gels, e.g., toothpastes or gels, including the composition of the invention.

The formulations and compositions described herein may also contain other ingredients such as antimicrobial agents or preservatives.

Exemplary Genes Useful in Vectors

A gene delivery vector may be designed to express any open reading frame, including but not limited to a therapeutic protein capable of preventing, inhibiting, stabilizing or reversing an inherited or noninherited genetic defect in metabolism, immune regulation, hormonal regulation, enzymatic or membrane associated structural function, or a prophylactic protein. Diseases which are amenable to treatment by a gene delivery vector of the invention include but are not limited to cystic fibrosis, Parkinson's disease, thalassemia, phenylketonuria, Lesch-Nyhan syndrome, severe combined immunodeficiency (SCID), Duchenne's Muscular Dystrophy, inherited emphysema, hypercholesterolemia, adenosine deaminase (ADA) deficiency, β-globin disorders, alantitrypsin (AAT) deficiency, hemophilia A, hemophilia B, Gaucher's disease, storage disease mucopolysaccharidosis type VII, hereditary lactose intolerance, diabetes, and leukemia, and the therapeutic gene may encode factor VIII, factor IX, factor V, adenosine deaminase, e.g., to treat leukemia arising from retroviral insertion, lactase, β-glucuronidase, antithrombin III, protein C, prothombin, or thrombomodulin.

In addition the vectors can be used to produce anti-sense nucleic acids in cells. Antisense therapy involves the production of nucleic acids that bind to a target nucleic acid, typically an RNA molecule, located within cells. Antisense therapy generally employs oligonucleotides that are complementary to mRNA molecules (“sense strands”) which encode a cellular product. Exemplary modes by which sequences can be targeted for therapeutic applications include: blocking the interaction of a protein with an RNA sequence (e.g., the interaction of RNA virus regulatory proteins with their RNA genomes); and targeting sequences causing inappropriate expression of cellular genes or cell proliferation (e.g., genes associated with cell cycle regulation; genetic disorders; and cancers (protooncogenes)). Exemplary potential target sequences are protooncogenes, oncogenes/tumor suppressor genes, transcription factors, and viral genes.

In addition, the vectors of the present invention can be used to deliver DNA sequences encoding catalytic RNA molecules into cells. For example, DNA sequences encoding a ribozyme of interest can be cloned into a vector of the present invention. Such a ribozyme may be a hammerhead ribozyme capable of cleaving a viral substrate, or an undesirable messenger RNA, such as that of an oncogene. The DNA-encoding ribozyme sequences can be expressed in tandem with tRNA sequences, with transcription directed from, for example, mammalian tRNA promoters.

Thus, exemplary gene products of interest for use with the vectors of the invention include but are not limited to, DNA sequences which code for an antisense or ribozyme sequence such as one to HIV-REV or a BCR-ABL sequence, code for proteins such as transdominant negative mutants which specifically prevent the integration of HIV genes into the host cell genomic DNA, replication of HIV sequences, translation of HIV proteins, processing of HIV mRNA, or virus packaging in human cells; code for wild-type conductance regulator (CFTR), wild-type p53, granulocyte macrophage colony stimulating factor (GM-CSF), as well as the LDL (low density lipoprotein) receptor, apo(a), phenylalanine hydroxylase, ornithine transcarboxylase (OTC), molecules which have superoxide dismutase activity, endothelial prostaglandin synthase, alpha-1 antitrypsin, erythropoietin, cytokines, e.g., IL-1, IL-2, IL-3, IL-4, IL-5, IL-6, IL-8, IL-9, IL-10, IL-11, IL-12, IL-13, IL-14, IL-15, and IL-18, alpha interferon, gamma interferons, G-CSF or tumor necrosis factors (TNFs), polypeptide or peptide hormones, blood clotting factors, phosphorylases, and kinases. Representative examples of antisense sequences include, but are not limited to, antisense thymidine kinase, antisense dihydrofolate reductase, antisense IL-1 receptor, antisense BER2, antisense ABL, antisense Myc, and antisense ras, as well as antisense sequences which block any of the enzymes in the nucleotide biosynthetic pathway, or antisense sequences for influenza virus, HIV, HSV, HPV, CMV, and HBV.

Further examples of genes to be used in the invention may include, but are not limited to, erythroid cell-specific genes, B-lymphocyte-specific genes, T-lymphocyte-specific genes, adenosine deaminase (ADA)-encoding genes, blood clotting factor-encoding genes, ion and transport channel-encoding genes, growth factor receptor- and hormone receptor-encoding genes, growth factor- and hormone-encoding genes, insulin-encoding genes, transcription factor-encoding genes, protooncogenes, cell cycle-regulating genes, nuclear and cytoplasmic structure-encoding genes, and enzyme-encoding genes, as well as genes encoding toxins or toxoids, e.g., diphteria toxoid and the like, to kill or otherwise damage and destroy targeted cells.

Prophylactic compositions may include a vector encoding a gene product for which is desirable to produce an immune response, e.g., a response to pathogens including viruses, e.g., gB of HSV, bacteria, yeast and fungi, or tumor antigens.

Methods of the Invention

The present invention also relates to a method of ameliorating the severity of a pathologic condition in a subject. The pathologic condition can be any condition in which it can be useful to introduce an expressible polynucleotide, including, for example, a congenital disease such as adenosine deaminase deficiency, or a cancer or other condition associated with an aberrant regulation of cell growth or death such as psoriasis or a hemangioma. As disclosed herein, the methods of the invention can be particularly useful for ameliorating a pathologic condition of the nervous system, for example, Parkinsons disease, Alzheimer's disease, or other neurodegenerative disorders. The expressible polynucleotide can encode a polypeptide that otherwise is absent or defective in the subject due, for example, to a congenital genetic defect; or can be an antisense molecule that reduces or inhibits the expression of a deleterious gene product such as an oncogene; or can be a polypeptide that induces apoptosis in cells in which it is expressed, for example, a caspase, or that kills cells expressing the polypeptide, for example, the HSV-TK gene product, which converts the nucleoside analog, ganciclovir, to an intermediate that is incorporated into elongating DNA and results in death of the cells (Moolten, 1986); or can be a polypeptide that has an immunostimulatory activity, for example, a cytokine or interferon. As such, a retroviral vector of the invention is useful as a medicament for treating any of a variety of pathologic conditions that can be ameliorated by expressing a polypeptide, antisense molecule, or the like, in a cell in a subject.

A subject that can be treated according to a method of the invention can be any organism such as a vertebrate subject, including a human; a domesticated or commercially valuable animal such as a cat, dog, horse, cow, pig, goat, bird, or fish; or the like. The severity of a pathologic condition can be ameliorated, for example by contacting a cell of the subject with the retroviral vector of the invention, which contains the desired expressible polynucleotide operatively linked to an appropriate transcriptional regulatory element, for example, a tissue specific element.

The vector containing the expressible polynucleotide also can be contacted with the cells of the subject in vivo. For in vivo treatment, the vector can be administered directly to the site of the target cell in the subject, or can be administered systemically or by shunting the vector through one or a few organs by cannulation of the appropriate blood vessels in the subject, such that the retroviral vector circulates to the site of the target cell. Generally, the retroviral vectors are formulated in a composition suitable for administration to the subject.

The invention will be further described by the following nonlimiting example.

### EXAMPLE

During the course of infection, human immunodeficiency virus-1 (HIV-1) enters and replicates in both CD4⁺ T cells and macrophages. As the infection progresses, the CD4⁺ T cell subset is depleted and the patient becomes susceptible to opportunistic infections. Previous studies indicate that cells of the monocyte/macrophage lineage play a number of important roles in HIV pathogenesis, including mediating HIV neuropathogenesis, serving as the initial infected cell populations, and acting as a viral reservoir throughout the course of infection (Flaherty et al., 1997; Ghorpade et al., 1998; Lambotte et al., 2000; van't Wout et al., 1994). However, the direct impact of HIV infection of macrophages in the context of a functional immune system is not known. It is desirable to be able to dissect the consequences of cell-specific infection in vivo. To achieve this, cell-specific HIVs need to be established. T cell specific HIV has been created either by mutating the vpr ORF preventing productive viral replication in macrophages (Eckstein et al., 2001) or by the elimination of C/EBP motifs in the upstream U3 (Henderson and Calame, 1997). HIV that replicates solely in macrophages does not exist to date. Restriction of HIV replication to macrophages by altering the envelope glycoprotein is not possible since R5-utilizing viruses readily enter both macrophages and primary CD4⁺ T cells (Grivel and Margolis, 1999; Malkevitch et al., 2001; Wu et al., 1997). Furthermore, modest changes in HIV envelope sequences alter tropism (Chesebro et al., 1996) via chemokine coreceptor utilization (Xiao et al., 1998) and evolution of coreceptor usage from CCR5 to CXCR4 is well documented in vivo (Donaldson et al., 1994; McNearney et al., 1993).

In contrast to the genetic variation found in HIV envelope sequence, the enhancer/promoter proximal region of the HIV-1 LTR that is critical for virus expression is relatively invariant (Blackard et al., 2000; Koken et al., 1992; Rousseau et al., 1997; Simm et al., 1996). The HIV enhancer/promoter proximal elements in most clades of HIV are composed of two NF-κB sites and three Sp1 sites; however, a third NF-κB site is found in many clade C viruses and lade E viruses may have the upstream NF-κB site replaced with a GABP site (Carr et al., 1996; Hunt et al., 2003; Verhoef et al., 1999). The NF-κB and Sp1 sites function well in both macrophages and T cells and were initially demonstrated to be relatively inconsequential to HIV tropism (Nagashunmugam et al., 1992; Pomerantz et al., 1991). More recently, a study suggested that the 5′ most Sp1 motif within the promoter proximal region may alter T cell specific transcription (McAllister et al., 2000). Upstream U3 sequences have been noted to be variable and can affect LTR activity in a cell-specific manner (Ross et al., 2001). For instance, two upstream motifs bind to members of the C/EBP family of transcription factors and are required for virus replication in macrophages, but not T cells (Henderson and Calame, 1997; Henderson et al., 1996, 1995). Conversely, upstream T cell specific elements such as NFAT (Shaw et al., 1988) and LEF (Corboy and Garl, 1997; Sheridan et al., 1995) are involved in regulating LTR activity in T cells, but not in macrophages.

Unlike the core enhancer of HIV, many other viral and cellular enhancer elements restrict expression in a cell-specific manner. As a consequence, the enhancer of some retroviruses play a critical role in controlling tropism (Celander and Haseltine, 1984; Rosen et al., 1985). For instance, the LTR enhancer of the lentivirus equine infectious anemia virus (EIAV) that is genetically hypervariable (Carpenter et al., 1991; Maury et al., 1997) is involved in the control of cell-specific EIAV expression (Maury, 1994; Maury et al., 2000). Previous work has demonstrated that three PU.1 binding motifs that are found in most strains of EIAV are required for LTR expression in primary macrophages. The EIAV PU.1 binding motifs are known to interact with the myeloid/B cell specific transcription factor PU.1(Carvalho and Derse, 1993b; Maury, 1994), whereas other enhancer motifs usually found within the EIAV LTR such as PEA-2 (AML-1), Oct, and CRE sites are important for expression in nonmyeloid cells (Maury et al., 2000).

The ability of LTR enhancer elements to control HIV cell tropism was explored. Also it was investigated whether HIVs containing a chimeric LTR replicate in a cell-specific manner. Myeloid-specific enhancer regions from EIAV were substituted for the wild-type HIV core enhancer/promoter proximal sequences. The HIV LTR elements both upstream and downstream from the enhancer region remained unchanged. Using this approach, a series of HIV/EIAV chimeric LTRs were generated that express in macrophages, but not T cells. When introduced into an infectious clone of HIV, these LTRs supported productive HIV replication in primary macrophages, but not purified CD4⁺ T cells or a T cell line. Thus, alteration of the HIV LTR can regulate production of infectious virions in a cell-specific manner.

Materials and Methods

Primary Cell Isolation and Maintenance

Human monocytes and CD4⁺ T cells were isolated from 300-350 ml of peripheral blood. Peripheral blood mononuclear cells (PBMCs) were isolated as previously described (Freundlich and Avdalovic, 1983). PBMCs were separated by centrifugation in lymphocyte separation medium (ICN) followed by a 40 minute incubation on flasks coated with gelatin and fibronectin to separate monocytes from the other mononuclear cells. Adherent monocytes were eluted with EDTA, washed five times, and plated at 1×10⁶ cells per well in 48-well plates for infectivity studies. Purified monocytes were differentiated for 5 days to generate monocyte-derived macrophages prior to HIV infections. MDMs were maintained in DMEM+10% FCS+10% human serum and 10 μ/mL penicillin and 10 μg/mL streptomycin. CD4⁺ T cells were separated from other peripheral blood lymphocytes (PBLs) by negative selection using Miltenyni magnetic beads per manufacturer's instructions following the monocyte adherence step. PBLs were incubated with monoclonal antibodies against CD8, CD14, CD16, CD19, CD36, CD56, CD123, TCR g/d, and Glycophorin A followed by incubation with Anti-Biotin MicroBeads. Unbound cells were separated from bound cells by passing through two LS columns. Prior to PHA stimulation, two-color flow analysis on PBLs and the purified CD4⁺ T cell population was performed using anti-CD3/PE-Cy5 (No. 15-0038-72 eBiosciences) and anti-CD4/PE (No. 12-0049-73 eBiosciences) to determine the effectiveness of the CD4⁺ T cell purification. Routinely, CD4⁺ T cells became highly enriched by the procedure with greater than 90% of the cells being CD4⁺ and CD3⁺. CD4⁺ T cells were treated with 5 μg/ml of PHA for 2 days. Activated cells were plated in a 24-well cluster plate at 10⁶ cells/well in RPMI with 10% FCS, 10 μ/mL penicillin, 10 μg/mL streptomycin, and 4 μ/ml of recombinant IL-2.

Cell Lines Used

Jurkat clone E6-1 and HEK 293 cells were obtained through the AIDS Research and Reference Reagent Program (Graham et al., 1977; Weiss et al., 1984). HeLa37 cells were a kind gift from Dr. David Kabat (Platt et al., 1998). Jurkat cells were maintained in RPMI 1640 with L-glutamine, 10 μ/mL pen/10 μg/mL strep, and 10% FCS. HEK 293 and HeLa37 cells were maintained in DMEM×10% FCS and 10 μ/mL pen/10 μg/mL strep. The canine macrophage cell line, DH82 (ATCC CRL 10389), was maintained in DMEM×10% FCS, 10 μ/mL pen, and 10 μg/mL strep.

HeLa37 clone 7 cells were generated by transfecting HeLa37 cells with the retroviral vector PU.1/LXSN that expresses human PU.1. Transfected cells were selected with 1 mg/ml of G418 for 2 weeks and colonies were biologically cloned, grown, and analyzed by electrophoretic mobility shift assays for expression of PU.1.

LTR-CAT Construction

For construction of chimeric LTRs, the pCMlu HIV LTR was used that contained a unique MluI restriction site in place of the enhancer/promoter proximal region of the HIV LTR (Lin et al., 1995). The pCMlu LTR was cloned into pCAT-BASIC (Promega) with XhoI and KasI sites that had been inserted into the vector and named pCMlu. The enhancer region from MT, an EIAV field isolate (Maury et al., 1997), was PCR amplified using the primers MLU/EIA137 and MLU/EIA172C′ and was cloned into pCMlu to form pMT (Table 1). Transcription factor binding motifs amplified from the MT LTR included three PU.1 motifs, a CRE, Oct, and Lvb motifs (Maury, 1994; Maury et al., 2000). The enhancer from the modified EIAV LTR, 3PU1 (R. A. M. Hines, submitted for publication), that retained only the 3 PU.1 sites, was amplified using oligonucleotides MLU/EIA127 and MLU/EIA172C′ and was cloned into pCMlu to produce p3PU1 (Table 1). The original 3PU1 LTR that was used as a template for amplification and generation of the 3PU1-based enhancers was generated from the MT LTR by site-directed mutagenesis of the Lvb, Oct, and CRE sites. Amplifications of the enhancers consisted of 24 cycles: 1 minute at 92° C., 30 s at 50° C., and 30 s at 72° C. All PCR products were cloned into pGEM-T (Promega) and sequenced. Correct clones were digested with MluI and the enhancers were cloned into pCMlu using the unique MluI restriction site. Insertion of the 3PU1 enhancer elements into the MluI restriction site in the antisense orientation resulted in 3PU1R, while multiple inserts are contained in 3PU13X.

TABLE 1
Oligonucleotides used in study

| | |
|---|---|
| Mlu/EIAV 137 | 5′-ACGCGTCAATATCCTGTAGT-3′ (SEQ ID NO:58) |
| Mlu/ELAV 127 | 5′-ACGCGTGTAGTTCCTCAATA-3′ (SEQ ID NO:59) |
| Mlu/ELAV 172C′ | 5′-ACGCGTAACAGGAACTTAAC-3′ (SEQ ID NO:60) |
| MLU389 | 5′-ACGCGTCCCAAGATTTCC-3′ (SEQ ID NO:61) |
| MLU398C′ | 5′-ACGCGTCTGATCCTCTTCTTCC-3′ (SEQ ID NO:62) |
| HIV −264 | 5′-GAACACCAGCTTGTTACACCC-3′ (SEQ ID NO:63) |
| HIV +335C′ | 5′-TCTCTCTCCTTCTAGCCTCCG-3′ (SEQ ID NO:64) |
| HIV118Δ15 | 5′-CCTGTT CCT AGGGCATATAAGCAGCTCG T |
| | TTTTGCC-3′ (SEQ ID NO:65) |
| HIV167Δ15C′ | 5′-GGACAAGGATCCCGT ATATTCGT |
| | CGACGAAAAACGC-5′ (SEQ ID NO:66) |
| MCSFPU1 | 5′-GAGGATCAGCCT AGGGAGGAGGAAGCT |
| | CAGATGCTGC-3′ (SEQ ID NO:67)* |
| MCSFPU1C′ | 3′-CT CCTAGT CGGATCCC T CCT CCTT |
| | CGAGTCTACGACG-5′ (SEQ ID NO:68) |
| 3PU1Sp1 | 5′-CCTGTTACGGGGAGTGGCGAGCCCTAGGATGCTGC |
| | ATATAAGCAGC-3′ (SEQ ID NO:69)* |
| 3PU1Sp1C′ | 3′-GGACAATGCCCCTCACCGCTCGGGATCCTACGACGT |
| | ATAT TCGTCG-5′ (SEQ ID NO:70)* |
| MTmdbp | 5′-CATCGAGCTTGCTAGCTGT |
| | TGCTAGGCAACAATATCCTG TAGTTCC-3′ (SEQ ID |
| | NO:71)* |
| MTmdbpC′ | 3′-GTAGCTCGAACGATCGACAACGATCCGTTGTTATAG |
| | GACATCAAGG-5′ (SEQ ID NO:72)* |
| 3PU1mdbp | 5′-CATCGAGCTTGCTAGCTGTTGCTAGGCAAGTAGTTC |
| | CTC-3′ (SEQ ID NO:73)* |
| 3PU1mdbpC′ | 3′-GTAGCTCGAACGATCGACAACGATCCGTTCATCAA |
| | GGAG-5′ (SEQ ID NO:74)* |
| For electro- | |
| phoretic | |
| mobility shift | |
| assays (only | |
| sense strand | |
| is shown): | |
| 124 | 5′-TCCTGTAGTTCCTCAATATAGTTCCGCATTTG-3′ |
| | (SEQ ID NO:75) |
| 136GAA | 5′-TCATTTGCGTGAAGCATTTCTGA-3′ (SEQ ID |
| | NO:76) |
| 91 | 5′-TAGCTCATGTTGCTAGGCAACTAAACCGCAAT-3′ |
| | (SEQ ID NO:77) |

*Underlined sequences are nucleotides that represent a unique restriction site that was used to facilitate plasmid screening; bolded sequences represent sequences that result in insertion of a new transcription factor binding motif.

LTR mutagenesis in which 15 nucleotides were deleted between the 3′ PU.1 motif and the TATA box was performed on pMT and p3PU1 using Stratagene's Quickchange Mutagenesis Kit as per the manufacturer's protocol using oligonucleotides HIV 118Δ15 and HIV167Δ15C′ (Table 1). Insertion of Sp1, MDBP, or both motifs into pMT and p3PU1 LTRs was also done using Quickchange Mutagenesis Kit (Stratagene) with the following oligonucleotides. To insert an Sp1 site, the oligonucleotides 3PU1 Sp1 and 3PU1 Sp1 C′ were used. To insert an MDBP motif, the following oligonucleotides were used for pMT:MTmdbp and MT mdbpC′ and for pC3PU1:3PU1mdbp and 3PU1mdbpC′ (Table 1). PCR-mediated mutagenesis consisted of one cycle of 30 seconds at 95° C. followed by 18 cycles of 30 seconds at 95° C., 1 minute at 55° C. and 10 minutes at 68° C. Insertion of the motifs into the same clone was done sequentially. DNA sequencing prior to use confirmed motif insertions.

Electroporations, Transfections, and CAT Assays

For Jurkat electroporations, 1×10⁷ cells were mixed with 10 μg of chimeric-LTR/CAT, 10 μg of CMV-β-gal, and 5 μg of either ssDNA or RSV-Tat in cytomix (Van Den Hoff et al., 1992). Jurkat cells were electroporated at 960 μF/310V using a Bio-Rad Gene Pulser II System. Cells were seeded into T25 flasks in RPMI+10% FCS and incubated for 44-48 hours at 37° C./5% CO₂. Cells were harvested by washing twice, followed by resuspension in 0.25 M Tris-HCl pH 7.8, and subjected to four to five freeze/thaw cycles. Cell lysates were normalized for β-gal activity using β-galactosidase enzyme assay system (Promega). All experiments were run in triplicate and repeated a minimum of two times.

HEK 293 cells were plated at 1.5-1.8×10⁵ cells in a six-well plate the day prior to transfection. Cells were transfected with 5 μg of LTR/CAT, 5 g of PU.1 expression vector, or the control vector pECE, 2.5 μg of RSV-Tat, or 2.5 μg of salmon sperm DNA and 1.5 μg of CMV-β-gal using the CaPO4 procedure (Graham et al., 1977). Cells were harvested by rinsing twice 44-48 hours post-transfection and lysed in 0.25 M Tris-HCl (pH 7.8). Cell lysates were normalized for β-gal activity using β-galactosidase enzyme assay system (Promega).

For DH82 transfections, 1.5-1.8×10⁵ cells were seeded into each well of a six-well plate the day prior to transfection. Cells were transfected with 1 μg of chimeric LTR/CAT, 1 μg of SV40-β-gal, and 0.5 μg of either ssDNA or RSV-Tat using GenePorter Transfection Reagent per the manufacturer's instructions (Gene Therapy Systems, San Diego, Calif.). Transfections were incubated for 44-48 hours in DMEM+10% FCS at 37° C./5% CO₂. Transfections were harvested by washing cells twice and either resuspended in 0.25 M Tris-HCl (pH 7.8) or subjected to four to five rounds of freeze/thawing. Cell lysates were normalized for β-gal activity using β-galactosidase enzyme assay system (Promega). All experiments were done in triplicate and repeated a minimum of two times.

Equivalent amounts of β-gal activity from transfected cell lysates were assayed for CAT activity. Cell lysates were incubated with ¹⁴C-chloramphenicol and acetyl coenzyme A as described by Gorman et al. (1983). Basal lysates were run for 1-2 hours and Tat-transactivated lysates were run for 5-30 minutes. Acetylated and unacetylated ¹⁴C-chloramphenicol were separated by thin-layer chromatography using silica thin-layer sheets and quantitated in an InstantImager (Packard Instruments). CAT activity was expressed as the % acetylation/m units of β-gal/hour.

Electrophoretic Mobility Shift Assays

Nuclear extracts were prepared as previously described (Maury, 1994) from Jurkat T cells, DH82 macrophage cell line, and primary monocyte-derived macrophages that had been allowed to differentiate in tissue cultures for 7 days. Protein concentrations were determined using Pierce Protein Assay System and extracts were frozen at −80° C. until used. Gel shifts were performed as previously described with 5-10 μg of nuclear extract (Maury, 1994). Briefly, nuclear extracts were incubated for 15 minutes at room temperature with 20,000 cpms of probe in the presence of 90 mM KCl, 1 mM MgCl₂ and 4 μg of poly(dIdC). Bound probe was separated from unbound probe on a 6% acrylamide gel in 1×TBE. Gels were fixed with methanol/acetic acid, dried, and autoradiographed overnight.

Virus Generation, RT Assays, and Infections

Infections were performed using p256, a dual-tropic molecular clone of HIV-1 (Chesebro et al., 1996), in which the 3′ LTR was replaced with a WT LTR or chimeric LTR. p256 was digested with XhoI (at 8887 in nef ORF) and NcoI (at 10,568 in the 3′ flanking sequence) and a polylinker containing an XbaI site was inserted. The WT and chimeric LTRs were removed from LTR/CAT vectors by digestion with XhoI and XbaI and cloned into the 3′ LTR position of p256 that had been digested with the same enzymes.

Viral stocks were obtained by transfecting HEK 293 cells with 5 μg of proviral DNA using the CaPO₄ procedure (Graham et al., 1977). For VSVG pseudotyping experiments, viral stocks were obtained from 1.5 μg of proviral DNA and 0.5 μg of VSVG-expressing plasmid that were cotransfected into HEK 293 cells using lipofection with Gene Porter per the manufacturer's instructions (Gene Therapy Systems). Cells were seeded at 1.5-1.8×10⁵ cells per well in a six-well plate the day prior to transfection. Supernatants were collected starting at 48 hours post-transfection and virus production was measured by RT assay and equivalent cpms were used to infect 6-day adherent monocyte-derived macrophages (d6 MDM) (10⁶ cells/well in a 48-well plate), CD4⁺ T cells (10⁶ cells/well in a 24-well plate), or Jurkat T cells (5×10⁵/well in a 24-well plate). RT assays were done as previously described (Willey et al., 1988).

HeLa37 cell or HeLa37 clone 7 cells (2.5×10⁴ cells/well in a 48-well plate) were infected with serial dilutions of virus and maintained for 40 hours. Cells were fixed in 75% acetone/25% water and immunostained for HIV antigens using human anti-HIV antisera (1:500), followed by HRP-conjugated goat anti-human IgG (1:500). AEC was used as the HRP substrate. Plates were dried and wells were counted for the number of HIV antigen-positive cells. Numbers of HIV antigen-positive cells were expressed as the infectious virions/ml of stock.

RT assays were performed by incubating 10 μl of cell-free supernatant with 50 μl of RT cocktail containing 50 mM Tris (pH 7.8), 75 mM KCl, 2 mM DTT, 5 mM MgCl₂, 0.05% NP-40, 5 μg poly(A) 4.71 μg poly(d) (T₁₂₋₁₈), and 10 μCi/ml [32P]-TTP. Mixtures were incubated at 37° C. for 2 hours and 10 μl was spotted onto DE81 paper (Whatman Cat. No. 3658-915). DE81 paper was washed three to five times in 3×SSPE and quantitated in an InstantImager (Packard Instruments).

PCR was performed on genomic DNA isolated from infected cells to verify that the 5′ LTRs contained the chimeric LTR. Approximately 25% of transfections resulted in LTR recombinations in which the 5′ LTR contained a WT enhancer/proximal promoter. Results from cultures where recombination was evident were not used in these studies. All viral stocks generated for infectivity studies were analyzed by DNA sequencing to ensure that the appropriate U3 elements were present in the 5′ LTR. PCR amplification consisted of 35 cycles: 1 minute at 94° C., 1 minute at 55° C., and 1 minute at 72° C. using the following primers: HIV (−264) and HIV (+335C′)′ (Table 1). The PCR products were purified using Millipore's Microcon filters and sequenced using the oligonucleotide primer HIV (−194) (Table 1).

Results

High-Level Expression of Chimeric LTRs was Detected in the Presence of the Macrophage-Specific Transcription Factor PU.1

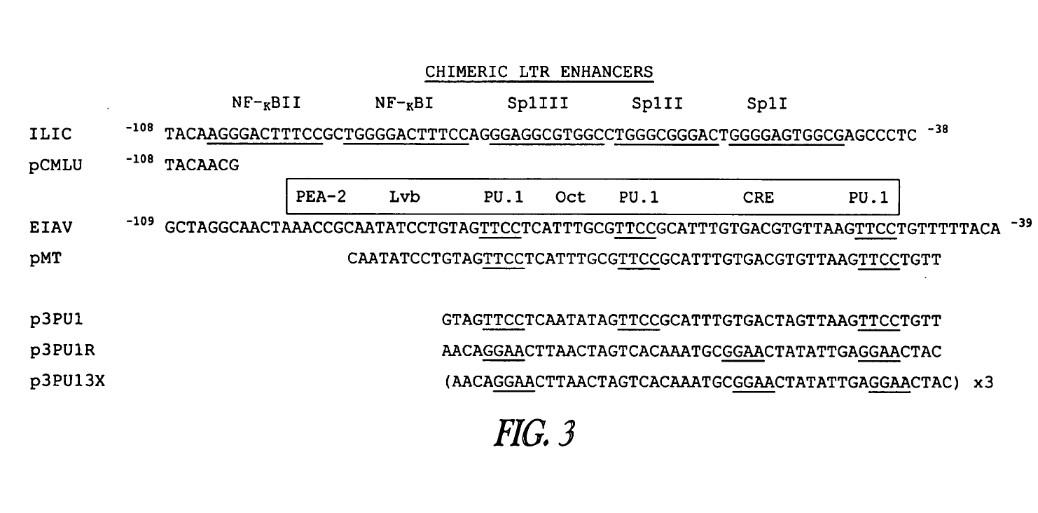

To investigate the ability of the LTR to confer cell tropism to HIV, chimeric HIV LTRs were created and tested for their ability to impart macrophage specificity to LTR activity through reporter gene assays and HIV replication. Viral enhancer regions that regulated expression in a macrophage-specific manner (Maury, 1994) were used as a basis for the studies. The enhancer elements were substituted for the HIV LTR enhancer/promoter proximal region while the remainder of the wild-type HIV LTR was maintained (FIG. 3). These enhancer regions included an EIAV sequence from a LTR obtained from a healthy, EIAV-seropositive horse in Montana (the MT series of LTRs) (Maury et al., 1997) and a modified EIAV enhancer that contained only the three PU.1 binding motifs (the 3PU1 series of LTRs). The 3PU1-based enhancers were designed to only contain motifs that interact with the myeloid/B cell-specific transcription factor PU.1 and consequently might be expected to have excellent macrophage specificity of expression. The 3PU.1 enhancer was tested for activity in the wild-type (3PU1) and reverse orientations (3PU1R). In addition, a triplication of the PU.1 enhancer region containing nine motifs in the reverse orientation (3PU13X) was analyzed for expression. 3PU13X that consisted of a concatemerized enhancer region containing nine PU.1 motifs was tested for activity since an earlier study suggested that PU.1 site concatemerization enhanced myeloid-specific transcription (Zhang et al., 1994).

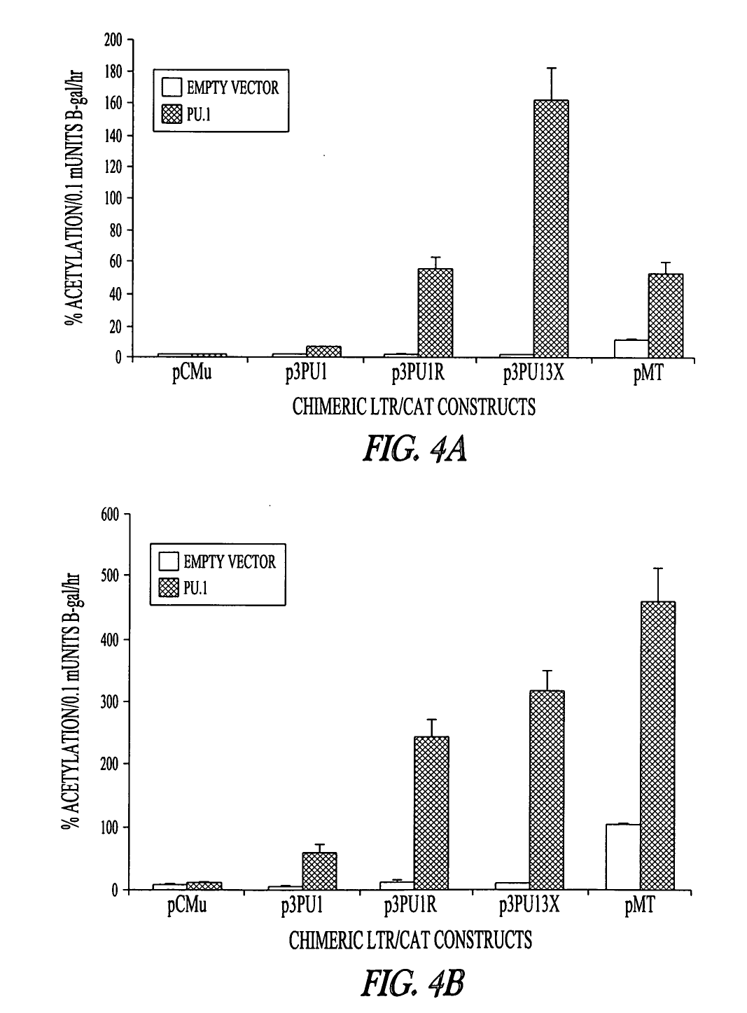

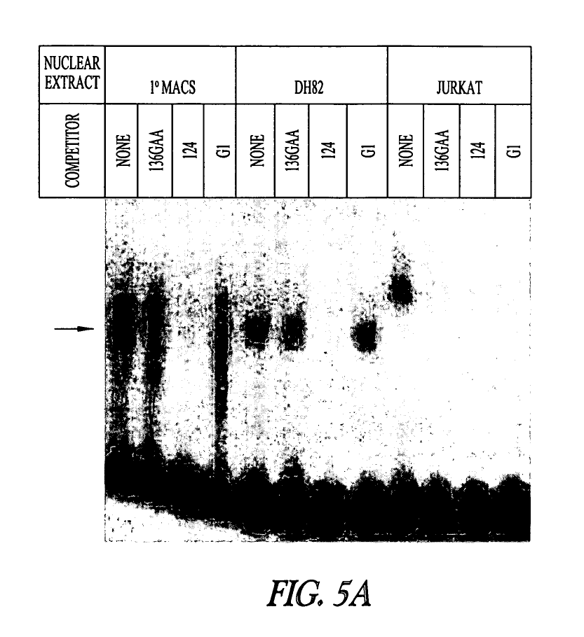

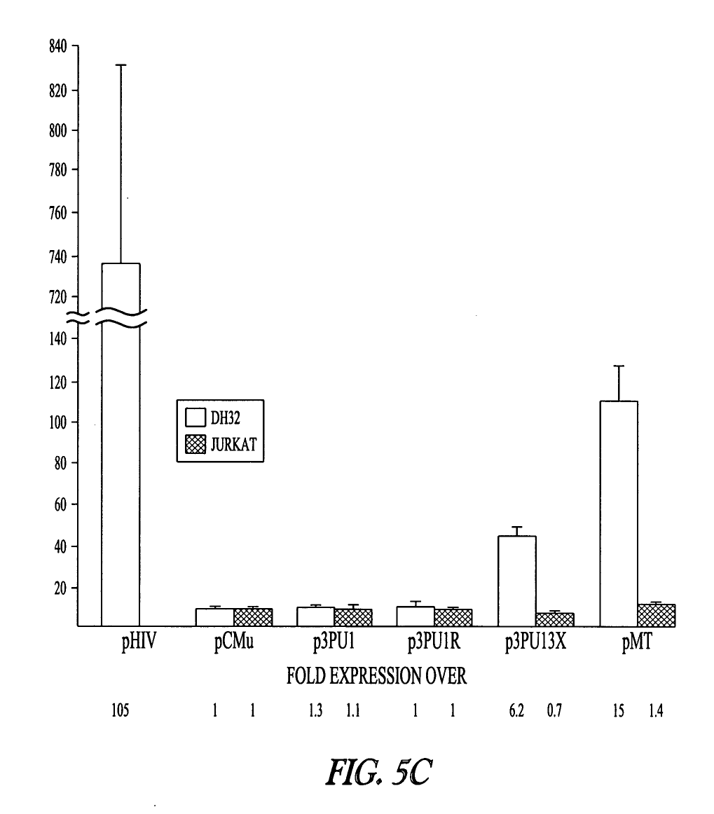

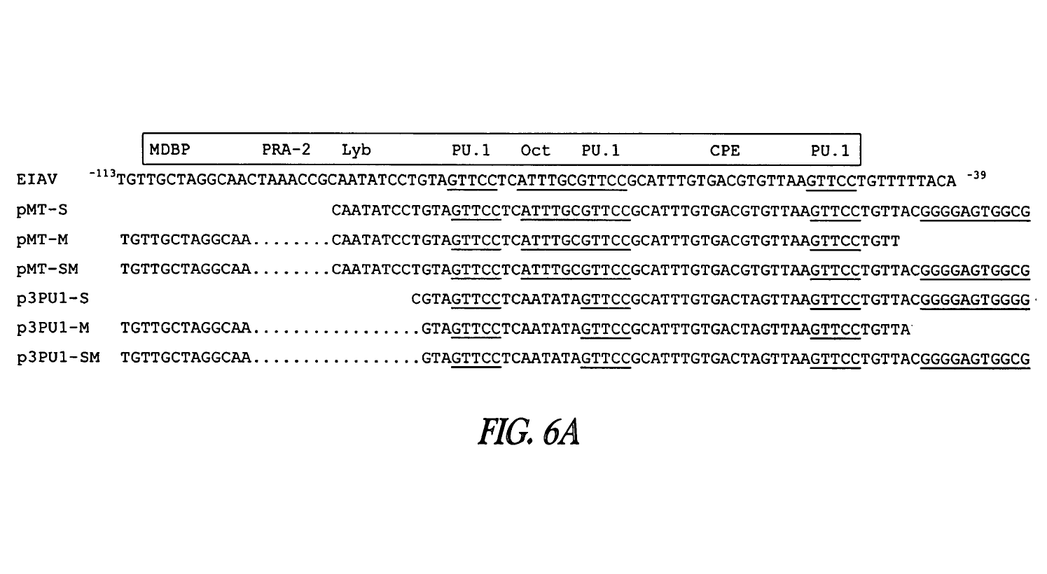

Initially, the strength and specificity of the chimeric enhancer region within the context of the HIV LTR was determined by performing transient transfection reporter gene assays. Because the chimeric viral enhancers contained motifs that had previously been shown to bind the macrophage and B cell specific transcription factor PU.1, initial studies were performed in either untransfected HEK 293 cells that do not express PU.1 or HEK 293 cells that transiently overexpressed PU.1 (FIG. 4). As anticipated, the chimeric LTRs 3PU1, 3PU1R, and 3PU13X that contained enhancers composed only of PU.1 sites required the exogenous PU.1 protein for expression in 293 cells. The pMT chimeric LTR was active in the absence of exogenous PU.1 but was enhanced approximately five-fold in basal and HIV Tat-transactivated studies in the presence of PU.1. The MT LTR activity observed in the absence of PU.1 overexpression was likely due to the presence of the additional transcription factor binding motifs, Lvb, Oct, and/or CRE, as 3PU1 does not contain these motifs and exhibited no basal activity in the absence of PU.1. The pCMlu LTR in which the enhancer/promoter proximal sequences were deleted and replaced with a MluI restriction site has previously been shown to be nonfunctional in T cells (Leonard et al., 1989) and was found to be inactive in these studies as well. These findings indicated that chimeric LTR activity required the inserted transcription factor motifs and was not simply due to the presence of upstream HIV-derived motifs such as the C/EBP sites. Additionally, consistent with a previous report (Lodie et al., 1998), these findings indicate that positive-acting PU.1 sites are not present in the pCMlu LTR. The 3PU13X chimeric LTR had slightly higher levels of basal activity than MT, but these higher basal levels did not result in the highest levels of activity in the presence of HIV Tat. In the presence of HIV Tat, the MT LTR was consistently the strongest chimeric enhancer. Since Tat transactivation is required for HIV replication, the initial findings suggested that the MT LTR might serve as the best chimeric LTR for supporting viral replication. Furthermore, these initial studies demonstrated that many of the chimeric LTRs were functional and might be cell specific since they required PU.1 for expression.

Expression of the Chimeric LTRs were Macrophage Specific

The chimeric LTR/CAT constructs were next tested for activity and specificity using an adherent canine macrophage cell line, DH82, which supports EIAV viral replication (Hines and Maury, 2001) and Jurkat T cells. DH82 cells were chosen for these experiments because the cells contain abundant PU.1 and are one of the few transfectable cell lines that have many properties of mature macrophages (Hines and Maury, 2001; Wellman et al., 1988). Previous studies investigating HIV LTR/reporter gene activity in myeloid-derived cell lines have utilized the murine macrophage cell line RAW264.7 (Lodie et al., 1998) or several human promonocytic cell lines that require pharmacological manipulation for maturation. Neither the murine nor the human myeloid lines are ideal for HIV LTR reporter gene studies. HIV Tat does not transactivate the HIV LTR in murine cells due to the inability of murine cyclin T1 to functionally interact with HIV Tat (Bieniasz et al., 1998; Garber et al., 1998). Problems with the human lines include the poor transfectability of these cells and the need to treat these cells with retinoic acid to establish an adherent macrophage phenotype. Since mature tissue macrophages are the target of HIV infection in vivo (Rich et al., 1992) and canine cyclin Ti functionally interacts with HIV Tat (Taube et al., 2000), a model cell that did not need to be pharmacologically manipulated to achieve a differentiated state was utilized.

The presence of PU.1 in macrophage, but not T cell nuclear extracts, was verified by electrophoretic mobility shift assays (EMSAs) using a probe derived from the EIAV enhancer (oligo 124 in Table 1) that contains the 5′ and middle PU.1 sites from the EIAV enhancer (FIG. 5A). Gel retardation of the probe was observed with all nuclear extracts. However, specificity of binding was only observed with monocyte-derived macrophage (MDM) and DH82 nuclear extracts. Approximately 200-fold excess of unlabeled specific competitor oligonucleotide (oligo 124) eliminated binding of the bands retarded by either MDM or DH82 nuclear extracts, whereas nonspecific oligonucleotides (136GAA and 91) did not compete for this binding. Oligonucleotide 136GAA includes EIAV enhancer nucleotides −75 to −52 with the PU.1 motif contained within this region mutated from TTCC to TGAA. Oligonucleotide 91 includes EIAV enhancer nucleotides −120 to −88 that does not contain an PU.1 site. In contrast to the results with either of the macrophage nuclear extracts, the slowly migrating band shifted by Jurkat nuclear extracts was eliminated by both specific and nonspecific competitors, indicating nonspecific binding.

In DH82 cell transient transfections, chimeric LTRs expressed between two-(p3PU1 R) and nine-fold (pMT) more reporter gene activity than the enhancerless LTR (pCMlu) in the absence of Tat (FIG. 5B). By comparison, the WT HIV LTR/CAT was 5-fold more active than pMT and 40-fold more active than pCMlu. In the presence of HIV Tat, wild-type HIV LTR activity was enhanced approximately 100-fold over that of pCMlu, indicating that strong levels of HIV Tat-transactivation occurred in DH82 cells (FIG. 5C). Some but not all of the chimeric LTRs were responsive to HIV Tat transactivation in DH82 cells. For instance, p3PU13X had 6-fold higher activity than pCMlu and pMT LTR had a 15-fold higher activity. To try to enhance chimeric LTR activity in DH82 cells, transfected cells were treated with LPS that is known to increase expression from the HIV LTR (Bagasra et al., 1992; Clouse et al., 1989). No enhancement of activity was observed with LPS stimulation (data not shown). pCMlu which retained the C/EBP motifs and other upstream U3 sequences was inactive. This finding indicated that while at least one C/EBP motif is necessary for LTR activity in macrophages in the context of the wild-type HIV LTR (Henderson and Calame, 1997; Henderson et al., 1995, 1996), these sites are not sufficient in the absence of the enhancer/proximal promoter elements to allow viral transcription to occur. Whether the upstream C/EBP sites are required in the chimeric LTRs for macrophage specific expression was not explored in this study.

In parallel with the LTR/reporter gene studies in macrophages, Jurkat T cells were tested for their ability to support the expression of the chimeric LTRs. The WT HIV LTR/CAT had a basal activity of 2.5±0.8% acetylation/0.1 munit b-gal/h and a 163-fold increase with the addition of Tat (data not shown). The basal activity of chimeric LTRs in Jurkat T cells was slightly higher than values obtained with the pCMlu LTR (FIG. 5B), but no different than values found with the empty CAT vector (data not shown). Consistent with experiments in 293 cells demonstrating the need for PU.1 for chimeric LTR expression, the addition of a Tat-expressing plasmid did not enhance the activity of the chimeric LTR constructs in Jurkats (FIG. 5C). LTR/CATtransfected Jurkat cells were activated to determine if cellular activation would enhance chimeric LTR activity. Activation studies using PMA and PHA failed to enhance LTR activity (data not shown), indicating that T cells do not support expression of these chimeric LTRs, presumably due to the lack of the necessary transcription factors.

Optimization of Expression of the Chimeric LTRs

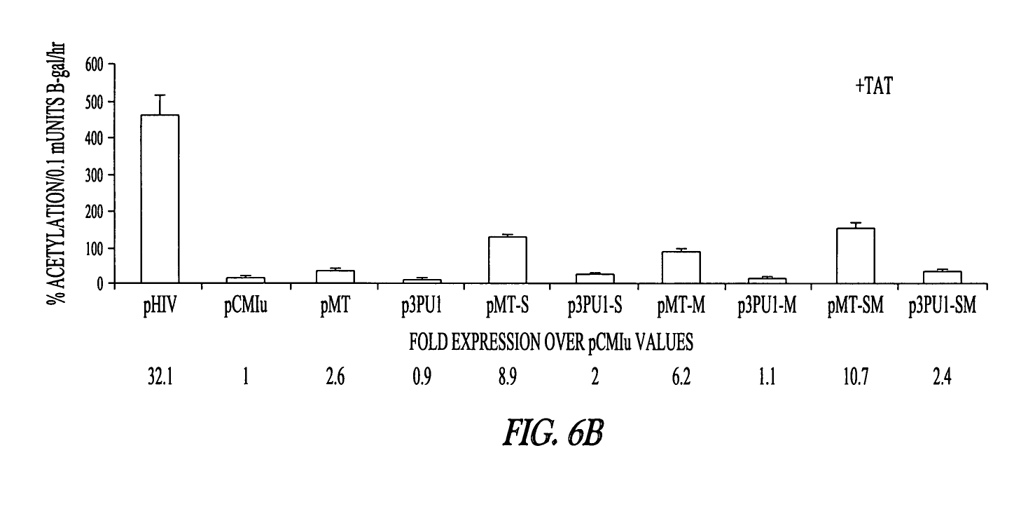

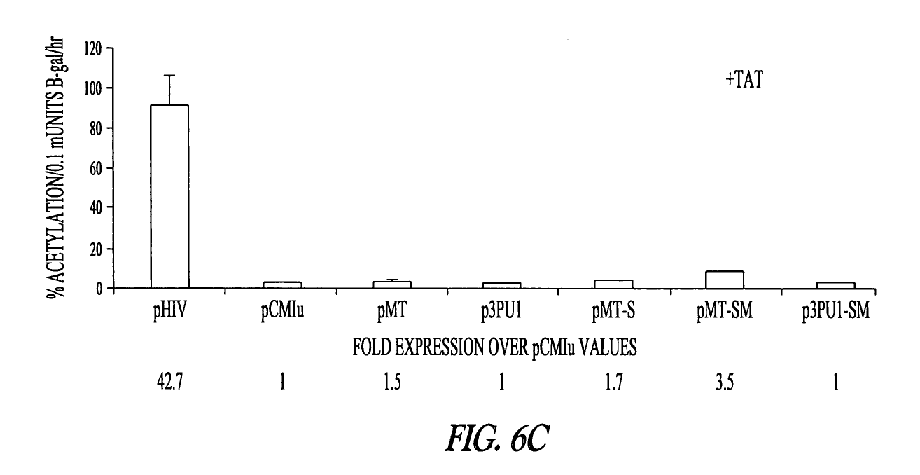

To determine if chimeric LTR activity could be enhanced while still maintaining cellular specificity, additional motifs were added to chimeric LTRs (FIG. 6A). A Sp1 site was included at the 3′ end of the enhancer. Previous work had demonstrated that an interaction between the promoter proximal Sp1 motif of the HIV LTR and Tat was necessary for optimal Tat responsiveness (Chun et al., 1998; Jeang et al., 1993). Additionally, a single Sp1 motif was shown to not be sufficient for HIV LTR activity in T cells (Ross et al., 1991) so insertion would not be predicted to alter cellular specificity. Because the spacing between the 3′ Sp1 motif and TATA box of HIV is important for Tat function and viral replication (Huang and Jeang, 1993), the Sp1 motif was inserted into the chimeric LTRs with the same spacing as seen in the WT HIV LTR. A second motif that was inserted in an upstream position was a methylation-dependent binding protein site (MDBP) that is present within the EIAV enhancer region. While the EIAV enhancer is hypervariable (Carpenter et al., 1991; Maury et al., 1997), there is a MDBP motif at the 5′ end of all known functional EIAV LTRs (Carvalho and Derse, 1993a; Maury et al., 1997). The function of this motif in the EIAV LTR remains unclear, but it is highly conserved, binds RFX proteins from a variety of cellular nuclear extracts, and enhances virus spread (Wendy Maury, unpublished observations). Because of the possible positive effects, the MDBP motif was inserted either alone or in combination with the Sp1 motif. Incorporation of either the Sp1 or the MDBP motif enhanced expression of the chimeric LTRs in DH82 cells in the presence of Tat (PMT-S and PMT-M) (FIG. 6B). However, a combination of the both Sp1 and MDBP motifs in conjunction with the MT enhancer (pMT-SM) consistently gave the highest levels of chimeric LTR expression.

To confirm that cellular specificity had not been compromised by the introduction of Sp1 and MDBP motifs, reporter gene studies were performed in Jurkat T cells. In basal studies, the inclusion of an Sp1 and/or MDBP motifs did not alter LTR function as all LTRs exhibited activities similar to the enhancerless LTR construct pCMlu (data not shown). In Tat-transactivated studies, inclusion the Sp1 and MDBP motifs within the pMT-SM LTR/CAT did enhance MT chimeric LTR activity 3.5-fold over background levels, suggesting the possibility that macrophage specificity was decreased by the addition of these motifs. Nonetheless, this level of activity was consistently less than the 11-fold increase upon Tat transactivation with the same construct in DH82 cells (FIG. 6C). As a further step to examine whether cellular activation would enhance basal levels of chimeric LTR function in Jurkat T cells, PHA/PMA were employed; however, there was no enhancement of LTR expression (data not shown).

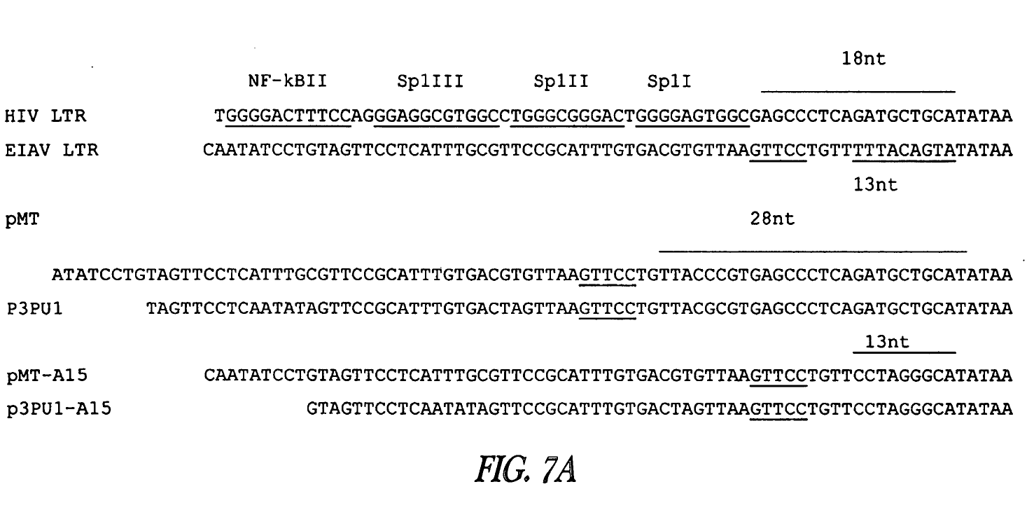

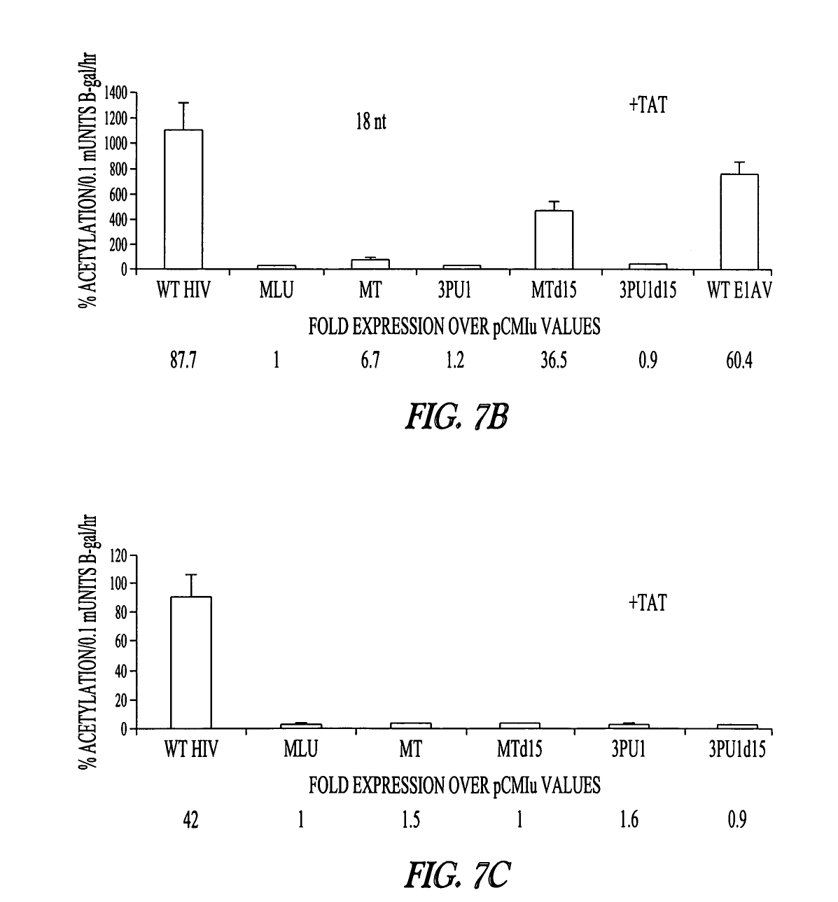

Spacing between enhancer and promoter sequences plays an important role in HIV LTR function (Huang and Jeang, 1993). Consistent with possible spacing constraints, the MT chimeric LTR exhibited increased Tat-transactivation when the Sp1 motif was incorporated with the proper spacing. Thus, the possibility that alteration of the enhancer/promoter spacing in the absence of additional transcription factor binding motifs could enhance the activity of the chimeric LTRs was tested. The spacing of the EIAV enhancer relative to the HIV TATA box in the MT LTR was decreased without introducing additional transcription factor binding motifs by introducing a spacing of 13 nucleotides between the core 3′ PU.1 motif and the TATA box (FIG. 7A). This 13 nucleotide spacing is found in the wild-type EIAV LTR. Spacing between the 3′ PU.1 motif and the TATA box was altered in both the MT and the 3PU1 chimeric LTRs. The MT-based LTR construct that contained wild-type spacing (pMT-Δ15) was 5.5-fold more active than the original pMT and had about 60% of the activity of WT EIAV/LTR CAT construct in DH82 macrophages (FIG. 7B). This LTR was one-third to one-half as active as the WT HIV LTR. Interestingly, decreased enhancer/promoter spacing in the p3PU1 (p3PU1-Δ15) construct only modestly enhanced activity and expression levels in these constructs remained low. When the altered enhancer/promoter spacing constructs were tested in Jurkat T cells, the altered spacing did not enhance activity from either the MT or the 3PU1-based LTRs, again indicating cellular specificity of the MT-based LTR (FIG. 7C).

Chimeric Viruses Replicated in Macrophages, But Not T cells

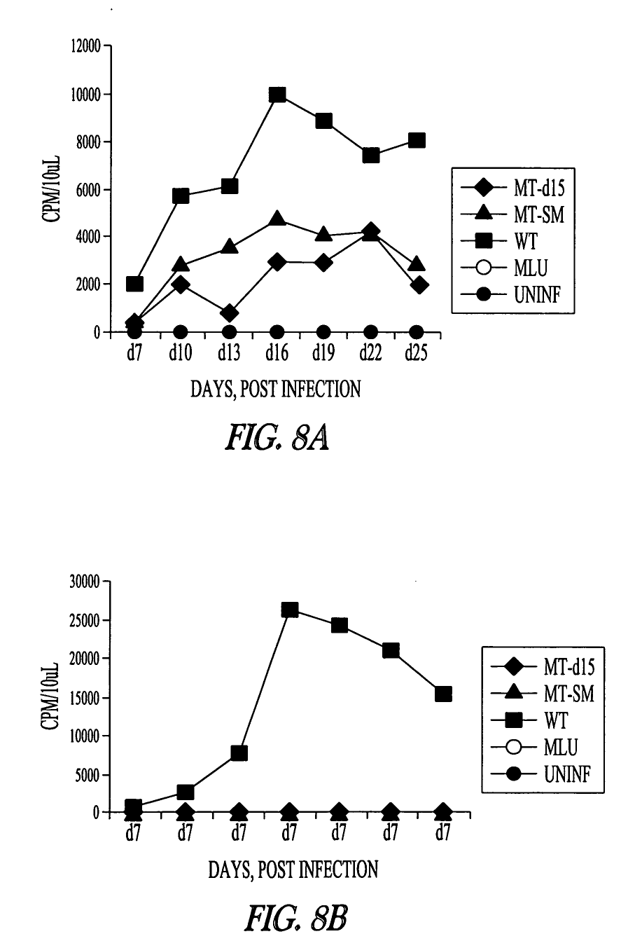

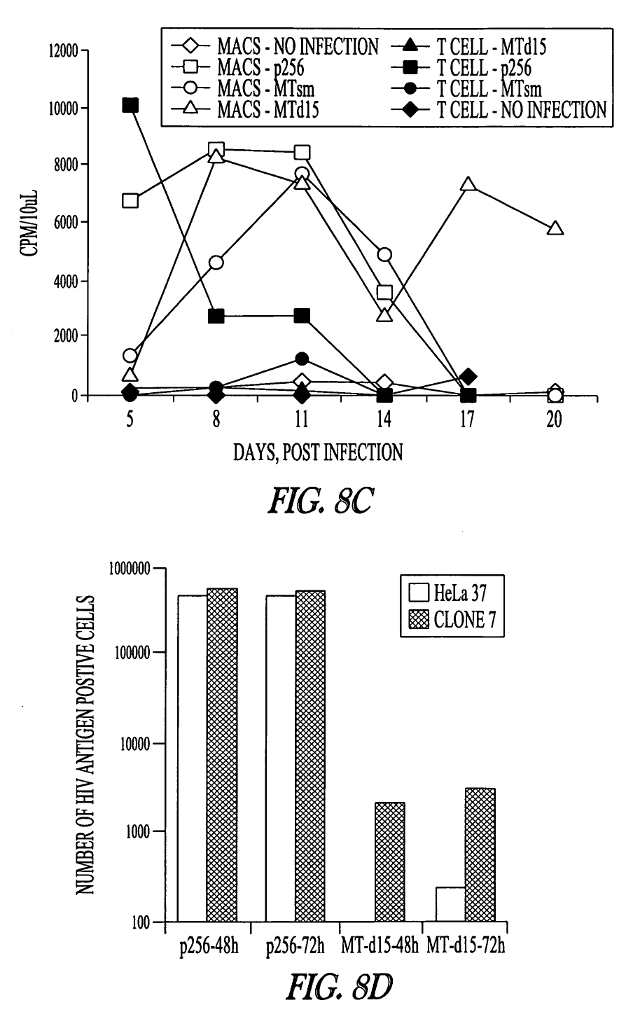

The true test of cellular specificity lies in the ability of the chimeric LTRs to direct viral replication in primary human macrophages but not CD4⁺ T cells. Since cellular specificity was being tested for restriction at the level of viral transcription, a dual-tropic molecular clone of HIV-1 p256 (Chesebro et al., 1996) was used. Chimeric LTRs were cloned into the 3′ LTR position because the 5′ U3 region containing the enhancer sequences is derived from the 3′ LTR during the process of reverse transcription in a newly infected cell. Chimeric LTRs that were functional in reporter gene studies were subcloned into the infectious clone and viral stocks were generated by collecting supernatants from transfected HEK 293 cells. Initial infections were performed in duplicate in MDMs and Jurkat T cells using equivalent levels of reverse transcriptase (RT) activity (cpms) in duplicate. MDMs were allowed to differentiate in tissue culture for 6 days prior to infections. As shown in FIG. 8A, the chimeric LTRs, MT-SM, and MT-Δ15 replicated in MDMs, resulting in about half the level of supernatant RT activity observed with WT HIV virus. In the same experiment, the chimeric LTRs 3PU1, 3PU1-SM, 3PU1-Δ15, 3PU1R, and 3× failed to produce detectable RT activity (data not shown). The inability of the 3PU.1-based viruses to replicate was somewhat surprising since some of these LTRs consistently gave detectable levels of CAT activity. However, EIAV studies within the lab have demonstrated that the three PU.1 sites in the absence of the other transcription factor binding motifs found in the EIAV enhancer is not sufficient to drive EIAV replication (data not shown). The pCMlu LTR also failed to produce virus in this and similar experiments, indicating that virus replication required the inserted enhancer sequences. In contrast, Jurkat T cells (FIG. 8B) or H9T cells (data not shown) were unable to support virus production when infected with virus containing the chimeric LTRs, while infections with wild-type p256 were readily detectable in the same experiment. At the termination of the experiment, genomic DNA from infected MDM cultures was isolated and the 5′ LTR was PCR amplified using U3 and gag-specific primers. For each infection, the 5′ LTR was verified by sequencing and shown to be the appropriate LTR (data not shown). Viral stocks of MT-SM and MT-Δ15 were repeatedly passaged in MDMs and supernatants from infected MDMs routinely gave supernatant RT values approximately half that of wild-type HIV. The LTRs present in viral stocks over the passages were verified by PCR amplification of LTR/gag DNA from infected MDM cultures. Only unaltered chimeric LTR sequences were recovered from chimeric stocks during passage, indicating that the chimeric LTRs were stable and able to support multiple rounds of HIV infection in macrophages.

Additional infectivity studies were performed in MDMs and primary CD4⁺ T cells using VSVG pseudotyped viral stocks. Pseudotyped stocks were used in these studies to eliminate any bias in receptor utilization. Supernatants containing equivalent quantities of cpms were added to either MDM or CD4⁺ T cell cultures and RT levels within supernatants from the infected cultures were monitored over 3 weeks (FIG. 8C). Consistent with experiments described above, pseudotyped MT-SM and MT-Δ15 replicated in MDMs but not primary CD4⁺ T cells from the same donor. In total, the present findings demonstrated the cellular specificity of the chimeric virus replication.

As a further test to determine whether PU.1 was required for replication of the chimeric viruses, stocks of parental p256 or pMT-Δ15 were added to either HeLa37 cells or HeLa37 clone 7 cells. HeLa37 cells stably expressed CD4 and CCR5 (Platt et al., 1998) as well as endogenous CXCR4. HeLa37 clone 7 cells were transduced with a retroviral vector (LXSN) that expressed human PU.1 and clone 7 was selected for use because of its high level of PU.1 expression (data not shown). Cells were infected in parallel with serial dilutions of virus, fixed at 40 hours postinfection, and immunostained for HIV antigens. The number of infected cells were quantitated and expressed as HIV positive cells/ml of virus stock. The number of infectious wild-type HIV virions detected HeLa37 and HeLa37 clone 7 cells were equivalent, while the PU.1 expressing HeLa37 cells supported more than log higher levels of MT-Δ15 viral expression (FIG. 8D). The detection of a small number of infected HeLa37 with the MT-Δ15 virus may be due to the ability of a small percentage of HeLa37 cells to support chimeric virus expression as a result of the presence of the Lvb, Oct, and CRE motifs that are present in the MT enhancer. This population of HIV antigen-positive cells in the MT-Δ15/HeLa37 cells were lost over several passages, whereas HIV antigen positivity was maintained in the MT-Δ15/HeLa37 clone 7 cells (data not shown). In addition, infectious virions present in the supernatants of the MT-Δ15/HeLa37 clone 7 cells could be passaged on to fresh HeLa37 clone 7 cells, but not to HeLa37 cells (data not shown). This data suggested that a small amount of viral transcription can occur from the MT-Δ15 in the absence of cellular PU.1; however, PU.1 is needed for detection of virus replication.

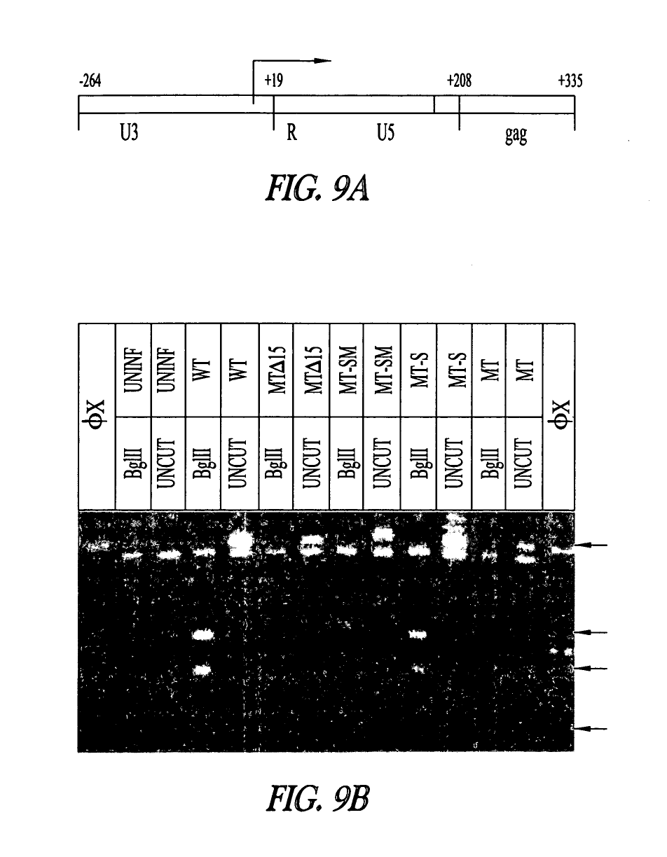

Since chimeric virus replication was not detected in T cells, to confirm that virus entry and reverse transcription were occurring, DNA was amplified from infected Jurkat cultures 6 days post-infection. As shown in FIG. 9, chimeric viral DNA was able to enter T cells and reverse transcribe. These findings indicated that the absence of detectable chimeric virus replication in T cells did not result from a block in the early life-cycle events.

Discussion

It was herein demonstrated that the cell tropism of HIV-1 can be altered by LTR enhancer/promoter proximal substitutions. HIV infectious chimeric viruses were prepared that were capable of replication in macrophages, but not T cells. The basis of the successful chimeric constructs was the enhancer region from the lentivirus EIAV. Previously, it was demonstrated that the three PU.1 motifs that interact with the macrophage/B cell specific transcription factor PU.1 are necessary for EIAV LTR activity in primary equine macrophages (Maury, 1994). Presumably the PU.1 motifs are responsible for the macrophage-specificity of the chimeric HIVs as demonstrated by studies in the PU.1 expressing HeLa cell line. Within the MT-based chimeric LTRs, not only were three PU.1 sites present, but also Lvb, Oct, and CRE motifs were present (Maury et al., 1997). The relative importance of these additional motifs for optimal expression was not explored in this study. However, as demonstrated by the 3PU.1 series of chimeric HIV LTRs, three PU.1 sites in the absence of the other EIAV transcription factor binding motifs were not sufficient to support chimeric HIV replication.

Interestingly, a series of chimeric LTRs based on the macrophage-specific cellular enhancer, MCSF-R, was not transcriptionally active (data not shown). The absence of activity from the MCSF-R enhancer may be due to the presence of the relatively strong upstream negative regulatory elements (NRE) within the HIV LTR that would need to be overcome by enhancer sequences. Alternatively, the MCSF-R enhancer may require additional motifs for optimal activity and/or this enhancer may not work in the context of the HIV promoter.

A previous study demonstrated that transient overexpression of PU.1 dramatically enhancer wild-type HIV LTR activity upon LPS stimulation, but not in the absence of LPS (Lodie et al., 1998). Extensive mapping performed in this study demonstrated that phosphorylated PU.1 stimulated LTR activity through the upstream NF-κB site. This site was deleted in the present constructs, but additional PU.1 sites were added. In light of these studies, it was surprising that the present constructs were not found to be responsive to LPS in DH82 cells (data not shown). The discrepancy in the present findings may be due to differences in the PU.1 sites used in the two studies or overexpression of PU.1 in the previous study.

A second generation of chimeric LTRs with enhanced expression was created based on the MT or the 3PU1chimeric LTRs. When a Sp1 and/or a MDBP motif was added to the chimeric LTR/CAT constructs, reporter gene activity was increased in both basal and Tat-transactivated reporter gene studies in macrophages and maximal activity was observed when both motifs were present (FIG. 6 and data not shown). The addition of the sites also resulted in higher titers of infectious virus for the MT-based LTRs while retaining cellular specificity. While enhanced LTR activity with the addition of the Sp1 site is consistent with earlier work, suggesting that the presence of a Sp1 site within the HIV enhancer increases Tat transactivation (Huang and Jeang, 1993; Jeang et al., 1993; Kamine and Chinnadurai, 1992; Kamine et al., 1991), the Sp1 site was not necessary for viral replication since MT-Δ15 was also infectious. Somewhat surprisingly, the modified 3PU1-based LTRs containing the Sp1 and MDBP sites were incapable of HIV replication.

An interesting aspect of the construction of the chimeric LTRs was the importance of spacing between the enhancer elements and the TATA box for optimal LTR activity. Previous work with the HIV-1 LTR has shown the importance of conserved spacing between the 3′ Sp1 motif and the promoter (Huang and Jeang, 1993). Spacing conservation between the 3′ PU.1 site and the TATA box in EIAV is also observed despite the highly heterogeneous nature of the EIAV enhancer (Maury et al., 1997). The initially studied chimeric LTRs did not retain appropriate enhancer/promoter spacing. Instead these constructs contained 28 nucleotides between the two transcriptional elements. When spacing was restored to the 13 nucleotides found in wild-type EIAV (MT-Δ15), LTR activity and viral infectivity was enhanced. Spacing between enhancer and promoter elements may have also played a role in the enhanced activity found in the Sp1 -containing LTRs. In these LTRs, in addition to the Sp1 site added, the enhancer/proximal promoter spacing was returned to the wild-type HIV spacing of 18 nucleotides.

Previous studies with HIV-1-based constructs have suggested that generation of a macrophage-specific HIV would be possible. Substitution of the HIV enhancer with heterologous enhancer regions from either other viruses or cellular genes in transgenic mice, lentiviral vectors, reporter gene constructs, and/or infectious viruses have been demonstrated. Numerous groups have placed a heterologous enhancer within the context of a lentiviral-based vector (Choi et al., 2001; Hamaguchi et al., 2000; Indraccolo et al., 2001; Iwakuma et al., 1999; Lotti et al., 2002; Moreau-Gaudry et al., 2001; Park and Kay, 2001; Ramezani et al., 2000). In some cases, this substitution has resulted in a cell tropism alteration of the vector, permitting expression of these constructs in cells such as endothelial cells or erythrocytes (Lotti et al., 2002; Moreau-Gaudry et al., 2001). HIV transgenic mice have been created that contain the Mo-MLV enhancer (nucleotides −365 to −79) in place of the two NF-κB motifs in the context of a HIV-1 provirus (Dickie et al., 1996). These authors demonstrated that in a transgenic setting viral expression could be restricted at the level of transcription in macrophages even following LPS stimulation. Chimeric HIV LTRs have also been created that were capable of producing replication-competent virus; however, these constructs did not alter the cellular tropism (Chang et al., 1993; Lin et al., 1995). The cytomegalovirus immediate early enhancer was used to replace the enhancer/proximal promoter of HIV-1 resulting in infectious virus that replicated in a subset of T cell lines; however, these studies did not look at virus replication in macrophages (Chang et al., 1993). Additionally, HIV-1 replication has been restricted to T cells by replacing the enhancer/proximal promoter motifs with the HTLV-1 Tax response element (TRE) limiting virus containing this heterologous enhancer to replicating only in HTLV-1 Tax expressing T cells (Lin et al., 1995). Finally, cellular enhancer sequences and other viral enhancers have been inserted into Mo-MLV LTR, resulting in an infectious murine virus (Feuer and Fan, 1990; Overhauser and Fan, 1985).

The present findings demonstrate for the first time that macrophage specificity of productive HIV replication can be mediated by LTR enhancer sequences. These constructs should provide tools for a better understanding of the role macrophages play in the pathogenesis of HIV infection in the absence of virus replication in T cells. For instance, exploration of the role of HIV infection of macrophages in bystander T cell death can be explored for the first time. Additionally, these constructs make it possible to determine if HIV-associated neuropathogenesis results from HIV infection of macrophages in the absence of T cell infection. Since primate lentiviral pathogenesis associated with macrophage infection appears to primarily manifest itself late in infection upon CD4⁺ T cell depletion (Igarashi et al., 2001), it is possible that HIV infection of macrophages in the presence of an intact immune response may have little or no pathogenicity associated with it. If that is the case, these constructs potentially could serve as a basis for a live, attenuated vaccine, stimulating an immune response without compromising the immune system. Additionally, these infectious clones may provide a gene therapy approach that specifically targets myeloid-lineage cells.

Bibliography

Akasaka et al., Cell. Mol. Biol., 45:555 (1999).
Antes et al., Biochemistry, 40:6731 (2001).
Bagasra et al., Proc. Natl. Acad. Sci. USA, 89:6285 (1992).
Bieniasz et al., EMBO J., 17:7056 (1998).
Blackard et al., Virology, 274:402 (2000).
Bollon et al., J. Clin. Hematol. Oncol., 10:39 (1980).
Botstein et al., Miami Wntr. Symp., 19:265 (1982).
Brigham et al., J. Liposome Res., 3:31 (1993)).
Broach, Cell, 28:203 (1982).
Broach, In: The Molecular Biology of the Yeast Saccharomyces: Life Cycle and Inheritance, Cold Spring Harbor Laboratory, Cold Spring Harbor, N.Y., p. 445-470 (1981).
Cai et al., Nature, 376:533 (1995).
Carpenter et al., J. Virol., 65:1605 (1991).
Carr et al., J. Virol., 70:5935 (1996).
Carvalho and Derse, J. Virol., 67:3885 (1993b).
Carvalho and Derse, J. Virol., 67:2064 (1993a).
Celander and Haseltine, Nature 312:159 (1984).
Chang et al., J. Virol., 67:743 (1993).
Chater et al., In: Sixth International Symposium on Actinomycetales Biology, Akademiai Kaido, Budapest, Hungary (1986), pp. 45-54.
Chesebro et al., J. Virol., 70:9055 (1996).
Choi et al., Cells, 19:236 (2001).
Chonn et al., Curr. Opin. Biotech., 6:698 (1995).
Chun et al., J. Virol., 72:2615 (1998).
Chung et al., Cell, 74:505 (1993).
Chung et al., Proc. Natl. Acad. Sci. USA, 94:575 (1997).
Clouse et al., J. Immunol., 143:470 (1989).
Corboy and Garl, J. Neurovirol., 3:331 (1997).
Curiel et al., Proc. Natl. Acad. Sci. USA, 88:8850 (1991).
Dickie et al., AIDS Res. Hum. Retroviruses, 12:1103 (1996).
Donaldson et al., J. Virol., 68: 5991 (1994).
Donze et al., Bioessays, 24:344 (2002).
Dunaway et al., Mol. Cell. Biol., 17:182 (1997).
Eckstein et al., J. Exp. Med., 194:1407 (2001).
Emery et al., Proc. Natl. Acad. Sci. USA, 97:9150 (2000).
Feuer and Fan, J. Virol., 64:6130 (1990).
Flaherty et al., J. Virol., 71:5790 (1997).
Flanagan et al., Mol. Cell Biol., 9:739 (1989).
Flanagan et al., Mol. Cell Biol., 12:38 (1992).
Fourel et al., EMBO J., 18:2522 (1999).
Freundlich and Avdalovic, J. Immunol. Methods, 62:31 (1983).
Garber et al., Genes Dev., 12:3512 (1998).
Geyer et al., Genes Dev., 6:1865 (1992).
Geyer et al., Cell & Mol. Sci., 59:2112 (2002).
Ghorpade et al., J. Virol., 72:3340 (1998).
Gorman et al., Science, 221:551 (1983).
Gosveld et al., Cell, 51:975 (1987).
Graham et al., J. Gen. Virol., 36:59 (1977)
Grivel and Margolis, Nat. Med., 5:344 (1999).
Gryczan, In: The Molecular Biology of the Bacilli, Academic Press, NY (1982), pp. 307-329).
Hamaguchi et al., J. Virol., 74:10778 (2000).
Henderson et al., Immunity, 5:91 (1996).
Henderson et al., J. Virol., 69:5337 (1995).
Henderson and Calame, Proc. Natl. Acad. Sci. USA 94:8714 (1997).
Hines and Maury, J. Virol. Methods, 95:47 (2001).
Huang and Jeang, J. Virol., 67:6937 (1993).
Hunt et al., Virus Genes, 26:49 (2003).
Igarashi et al., Proc. Natl. Acad. Sci. USA, 98:658 (2001).
Indraccolo et al., Blood, 98:3607 (2001).
Isner, Nature, 415:234 (2002)
Iwakuma et al., Virology, 261:120 (1999).
Izaki, Jpn. J. Bacteriol., 33:729 (1978).
Jeang et al., J. Virol., 67:6224 (1993).
John et al. Rev. Infect. Dis., 8:693 (1986).
Kamine and Chinnadurai, J. Virol., 66:3932 (1992).
Kamine et al., Proc. Natl. Acad. Sci. USA, 88:8510 (1991).
Kellum et al., Cell, 64:941 (1991).
Kellum et al., Mol. Cell Biol., 12:2424 (1992).
Kendall et al., J. Bacteriol., 169:4177 (1987).
Koken et al., Virology, 191:968 (1992).
Krebs et al., Mol. Cell, 1:301 (1998).
Lambotte et al., J. Acquir. Immune Defic. Syndr., 23:114 (2000).
Leboulch et al., EMBO J., 13:3065 (1994).
Ledley, Human Gene Therapy, 6:1129 (1995).
Leonard et al., J. Virol., 63:4919 (1989).
Lin et al., J. Virol., 69:7216 (1995).
Litt et al., EMBO J., 20:2224 (2001).
Lodie et al., J. Immunol., 161:268 (1998).
Lotti et al., J. Virol. 76:3996 (2002).
Malkevitch et al., Virology, 281:239 (2001).
Maniatis, In: Cell Biology: A Comprehensive Treatise, Vol. 3, Gene Sequence Expression, Academic Press, NY, pp. 563-608 (1980).
Maury et al., J. Virol., 71:4929 (1997).
Maury et al., Virology, 267:267 (2000).
Maury, J. Virol., 68:6270 (1994).
McAllister et al., Virology, 274:262 (2000).
McCune et al., Nucleic Acids Res., 22:4477 (1994).
McKnight et al., Proc. Natl. Acad. Sci. USA, 89:6943 (1992).
McNearney et al., Pediatr. Res., 33:36 (1993).
Miller, et al., FASEB Journal, 9:190 (1995).
Modin et al., J. Virol., 74:11597 (2000).
Moolten, Cancer Res., 46:5276 (1986).
Moreau-Gaudry et al., Blood, 98:2664 (2001).
Nagashunmugam et al., Pathobiology, 60:234 (1992).
Namciu et al., Mol. Cell. Biol. 18:2382 (1998).
Needleman and Wunsch, J. Mol. Bio., 48: 443 (1970).
Okayama, Molec. Cell. Bio., 3:280 (1983).
Osborne et al., J. Virol., 73:5490 (1999).
Overhauser and Fan J. Virol., 54:133 (1985).
Pannell et al., EMBO J., 19:5884 (2000);
Park and Kim, Mol. Ther., 4:164 (2001).
Pearson and Lipman, Proc. Natl. Acad. Sci. USA, 85:2444 (1988).
Phi-Van et al., Biochemistry, 35:10735 (1996).
Phi-Van et al., Mol. Cell. Biol., 10:2302 (1990).
Pikaart et al., Genes Dev., 12:2852 (1998).
Platt et al., J. Virol., 72:2855 (1998).
Pomerantz et al., J. Virol., 65:1041 (1991).
Ramezani et al., Mol. Ther., 2:458 (2000).
Reed-Inderbitzin et al., Virology, 314:680 (2003).
Remington's Pharmaceuticals Sciences, 17^{th} ed., Mack Publishing Company, Easton Pa. (1985).
Rich et al., J. Clin. Invest., 89:176 (1992).
Rivella et al., J. Virol., 74:4679 (2000).
Roseman et al., EMBO J., 12:435 (1993).
Rosen et al., J. Virol., 55:862 (1985).
Ross et al., J. Virol., 65:4350 (1991).
Ross et al., J. Virol., 75:1842 (2001).
Rousseau et al., AIDS Res. Hum. Retroviruses, 13:1611 (1997).
Ryan et al., Proc. Natl. Acad. Sci. USA, 86:37 (1989).
Sabatino et al., J. Biol. Chem., 275:28549 (2000).
Sabatino et al., Mol. Cell Biol., 18:6634 (1998).
Sadelain et al., Proc. Natl. Acad. Sci. USA, 92:6728 (1995).
Saitoh et al., EMBO J., 19:2315 (2000).
Sambrook et al., Molecular Cloning: A Laboratory Manual, third edition, Cold Spring Harbor Laboratory, (2001)).
Schnepp et al., Meth. Mol. Med., 69:427 (2002).
Schofield et al., British Med. Bull., 51:56 (1995).
Schreier, Pharmaceutica Acta Helvetiae, 68:145 (1994).
Scott et al., EMBO J., 14:6258 (1995).
Shaw et al., Science, 241:202 (1988).
Sheridan et al., Genes Dev., 9:2090 (1995).
Simm et al., AIDS Res. Hum. Retroviruses, 12:801 (1996).
Simmonds, P., J. Virol., 68:5991 (1994).
Smith and Waterman, Adv. Appl. Math., 2: 482 (1981).
Speck et al., Mo. Cell Biol., 7:1101 (1987).
Stief et al., Nature, 341:343 (1989).
Sun et al, Cell, 99:459 (1999).
Takada et al., Mol. Reprod. Dev., 57:232 (2000).
Taube et al., J. Virol., 74:892 (2000).
Tsukiyama et al., Mol. Cell Biol., 9:4670 (1989).
Tsukiyama et al., Mol. Cell Biol., 12:1286 (1992).
Van Den Hoff et al., Nucleic Acids Res., 20:2902 (1992).
van der Vlag et al., J. Biol. Chem., 275:697 (2000).
Van't Wout et al., J. Clin. Invest., 94:2060 (1994).
Verhoefet al., J. Virol., 73:1331 (1999).
Weiss et al., J. Immunol., 133:123 (1984).
Wellman et al., In Vitro Cell. Dev. Biol., 24:223 (1988).
Willey et al., J. Virol., 62:139 (1988).
Willoughby et al., J. Bio. Chem., 275:759 (2000).
Wu et al., J. Exp. Med., 185:1681 (1997).
Xiao et al., Virology, 240:83 (1998).
Zhang et al., Mol. Cell. Biol., 14:373 (1994).
Zhong et al., Proc. Natl. Acad. Sci. USA, 94:5219 (1997).

All publications and patents are incorporated by reference herein, as though individually incorporated by reference. The invention is not limited to the exact details shown and described, for it should be understood that many variations and modifications may be made while remaining within the spirit and scope of the invention defined by the statements.

What is claimed is:

1. A vector comprising a chimeric recombinant DNA molecule comprising one or more insulators, one or more transcriptional regulatory control sequences, and an open reading frame for a gene product, wherein at least one insulator is at least 10 nucleotides but no more than about 150 nucleotides in length and is 5′ to at least one transcriptional regulatory control sequence which is operably linked to the open reading frame and/or optionally 3′ to the open reading frame.

2. The vector of claim 1, wherein the transcriptional regulatory control sequence which is operably linked to the open reading frame is a promoter.

3. The vector of claim 2, wherein the promoter is a viral promoter.

4. The vector of claim 2, wherein the promoter is a cellular promoter.

5. The vector of claim 2, wherein the promoter is heterologous to at least one insulator.

6. The vector of claim 1, wherein at least one insulator is 5′ to transcriptional regulatory control sequences which include an enhancer and a promoter.

7. The vector of claim 6, wherein the enhancer is heterologous to the promoter.

8. The vector of claim 1 which is a viral vector.

9. The vector of claim 8 which is a retroviral vector, a lentiviral vector, or an adeno-associated virus vector.

10. The vector of claim 8, wherein one or more insulators are 3′ to the open reading frame.

11. The vector of claim 9, wherein at least one insulator is introduced into the 3′ LTR of a retroviral or lentiviral vector.

12. The vector of claim 9, wherein at least one insulator is introduced into the 5′ ITR and optionally the 3′ ITR of an adeno-associated viral vector.

13. The vector of claim 1, wherein at least one insulator comprises GTTGCTAGGCAAC (SEQ ID NO:31).

14. The vector of claim 1 wherein at least one insulator comprises a sequence of formula I: X₂RT^{m}YRYYX₁^{m}YRG^{m}YRAYX₃ (SEQ ID NO:32), wherein X₁ is A or is absent, R is a purine, Y is a pyrimidine, ^{m}Y is thymidine or 5-methylcytosine, and X₂ and X₃ are individually absent, a nucleotide, or a nucleotide sequence of 2 or more and up to about 140 nucleotides.

15. The vector of claim 1, wherein the open reading frame encodes a therapeutic gene product.

16. The vector of claim 1, wherein the open reading frame encodes a prophylactic gene product.

17. The vector of claim 1, wherein the open reading frame encodes a catalytic RNA.

18. A plasmid comprising the vector of claim 1.

19. A host cell comprising the vector of claim 1 or a plasmid comprising the vector.

20. The host cell of claim 19 wherein the vector is stably integrated into the genome of the mammalian cell.

21. The host cell of claim 19 wherein the vector is an adenovirus, lentivirus, adeno-associated virus (AAV), polyomavirus, herpes simplex virus, or retrovirus vector.

22. A method to alter the expression of an open reading frame in a cell, comprising contacting a cell with the vector of claim 1 or a plasmid comprising the vector so as to yield a cell which stably expresses the open reading frame at a level which is different than the level of expression in a corresponding cell having a corresponding vector or corresponding plasmid which lacks the one or more insulators.

23. A method to inhibit or treat a condition associated with aberrant expression of an endogenous gene product, comprising: contacting a mammal at risk of or having the condition, with an effective amount of the vector of claim 1, a plasmid comprising the vector, or a host cell comprising the vector or plasmid which vector, plasmid or host cell contains an open reading frame encoding at least a portion of a functional gene product, the expression of which in the mammal inhibits or treats at least one symptom of the condition.

24. The method of claim 23, wherein prior to contacting, the mammal lacks expression of the endogenous gene product.

25. The method of claim 23, wherein prior to contacting, the mammal overexpresses the endogenous gene product.

26. The method of claim 23, wherein prior to contacting, the mammal has reduced expression of the endogenous gene product.

27. A method to express an open reading frame in a vector which is stably associated with a mammalian cell comprising: stably introducing to the mammalian cell the vector of claim 1 or a plasmid comprising the vector so as to yield a stably genetically transformed mammalian cell which expresses the open reading frame.

28. The method of claim 27, wherein the gene product is a reporter protein.

29. The method of claim 27, wherein the gene product is a prophylactic or therapeutic gene product.
